(19) United States
(12) Patent Application Publication
(10) Pub. No.: US 2005/0136413 A1
(43) Pub. Date: Jun. 23, 2005

(54) Reagent systems for biological assays

(76) Inventors: Michael W. Briggs, Hampton, NH (US); Thomas A. Bunch, Painted Post, NY (US); Andrew M. Ferrie, Painted Post, NY (US); Xinying Xie, Painted Post, NY (US)

Correspondence Address: CORNING INCORPORATED, SP-TI-3-1, CORNING, NY, 14831, US

(21) Appl. No.: 10/745,835

(22) Filed: Dec. 22, 2003

(51) Int. Cl. C12Q 1/68

(52) U.S. Cl. 435/6

(57) ABSTRACT

A reagent system for printing, assaying, and processing nucleic acid microarrays is provided. The system comprises: a printing kit, which includes a nucleic acid spotting solution; and a hybridization kit, which includes a nucleic acid pre-hybridization solution, a nucleic acid hybridization solution, and first, second, and third wash reagents, wherein the respective constituent components of the printing and hybridization kits are stable and retain functional performance, when stored together at a temperature between about 10° C. to about 50° C. A background reducing agent or solution is also included. The present reagent solutions are optimized for use with glass substrates having preferably an amine-coating, such as GAPS.

### INTRODUCTION

Section I—Field of the Invention

The present invention pertains to reagent kits and their use for performing biological assays on nucleic acid-based microarrays. In particular, the invention relates to certain combinations and formulations of reagents that can enhance the results of nucleic acid hybridization assays, as well as are stable even when stored at ambient or higher temperatures.

Section II—Background

In recent years, the biological, pharmaceutical, and other research communities have recognized that microarrays are useful, high-throughput research tools to measure a variety of biological or biochemical interactions and functions. With widespread acceptance, the microarray format is likely to remain a key research tool into the foreseeable future. Applications for microarray technology will continue to expand in the areas of drug discovery and development, diagnostics, and basic research.

For instance, high-density arrays have become invaluable tools for drug researchers and geneticists to obtain information on the expression of genes. One may monitor changes in gene expression profiles or single nucleotide polymorphism (SNP) of genes of interest using a microarray containing nucleic acid analytes or probes. One kind of application for such arrays is to test whether target DNA sequences interact or hybridize with any of the probes on the array. According to conventional protocols, the testing procedure consists of printing and binding probe nucleic acid molecules onto a substrate to form a microarray. The substrate may be any size, but typically takes the form of a standard 1-inch×3-inch microscope slide. Generally, samples of nucleic acids, such as obtained from a patient, are tagged with a fluorescent marker. The target nucleic acids are allowed to interact with probes on the microarray for a specified period of time, followed by rinsing to remove unbound targets. If the target nucleic acid sample contains any complementary sequences to the known probe strands on the surface, hybridization occurs and is detected as fluorescence from the marker of the target nucleic acids bound to probes. The ratio of fluorescent intensity of genes from abnormal cells relative to a reference from normal cells at each spot on the high-density array provides the relative differential expression for a particular gene. The difference in the ratio implies that the genes are either turned on, “up-regulated,” or turned off, “down-regulated”, in the abnormal cells. For example, a researcher can compare the hybridization results of genes in a normal colon cell with those in a malignant colon cell using a single assay; thereby, determining which genes are being expressed or not expressed in the aberrant cell. The regulatory sites of genes may serve as key targets for drug therapy.

Alternatively, clinical and research laboratories are increasingly using DNA testing as a means to determine genetic risk factors for diseases like breast cancer, heart disease, Alzheimer's disease, etc. Simultaneous screening for many risk factors is possible by printing many “microdots” of DNA onto the same substrate, typically either a porous, organic membrane or a flat, non-porous glass slide to form a high-density array. A high-density array typically comprises between 2,000 and 50,000 probes, with the possibility of up to about 80,000 or 100,000 probes, each of a known and different sequence, arranged in a predetermined pattern on a substrate.

Performance of a nucleic acid array is influenced by two major factors: 1) retention of the immobilized probe nucleic acid sequences on the substrate, and 2) hybridization of the target sequence to the immobilized probe sequence, as measured by fluorescence emission from the bound, tagged target sequence. The nucleic acid probes must be retained on the surface of the substrate through a series of blocking, hybridizing, and washing or rinsing operations that are commonplace in DNA hybridization assays. Advances of recent years in substrate materials and surface chemistries have helped to improve attachment and retention of DNA or other biological molecules. Improvements with respect to hybridization techniques and reagents, on the other hand, have not advanced as significantly.

Currently, the market for microarray technology is divided mainly into two formats. One format involves so-called “pre-printed arrays,” on which the commercial vendor has already secured nucleic acid probes to an array substrate. A second format includes so-called “self-printed arrays,” on which the customer or final user prints his own array. Recent marketplace trends of consumer preference indicate movement toward greater use of self-printed arrays. The motivations for increased use of self-printed arrays are varied, but some include ease of use, wide flexibility or customizability, and cost savings. For instance, customers can select their own particular genes of interest to deposit onto the array, the type of genetic material (e.g., oligonucleotides, genomic DNA, and cDNA), and the density or design of the array, etc. The reason the ultimate user is attracted to self-printing is driven by the relatively inexpensive and developed printing technology in combination with the availability of pre-coated substrates, which makes creating nucleic acid arrays relatively less complex.

Whereas the trend for microarray formats is toward more self-printed arrays, the opposite trend is appearing for hybridization reagent solutions. Researchers who use microarrays are seeking to buy “complete solutions” of hybridization reagents rather than choose solution components separately, à la carte style. Reasons for this may include the fact that complete solutions have been quality tested empirically, and they provide standardization and instant expertise. These features afford the customer both friendlier method of use and higher data integrity. That is, researchers need not mix and prepare their own solutions, and expend time and resources developing optimal formulations. Moreover, customers can more easily compare results among different assay experiments.

Currently there are a number of vendors that provide solutions for membrane and glass-substrate type arrays. Even though the solutions these suppliers provide work well, a need still exists for an all in one reagent kit, which provides superior assay performance in terms of sensitivity, dynamic range and reproducibility; measurable increase in productivity and stability; and manufacturing excellence.

### SUMMARY OF THE INVENTION

The present invention provides, in part, an assembly or reagent system for performing biological assays. The system involves the use of at least one or more reagent kits or compositions adapted for various steps of an assay process. Each solution contains one or more components in a single medium. Some solutions, with two or more components, have a formulation that combines in a single medium various components, which heretofore have been considered to be incompatible with each other when stored together in a single vessel or common mixture. In the past, when more than one component is present in a single medium, adverse issues associated with storage stability typically arise. The conventional approach to avoid such issues is to store these reagents separately at different temperatures, physically separate from each other, or under different environmental conditions.

In contrast, the reagent solutions according to the present invention can be stored in a single medium or mixture combination over a wide range of temperatures and under much less stringent conditions. The reagents can be stored at temperatures of about −20° C. to about 60° C. and still maintain compositional stability and retain their respective functional performance. All kit components can meet optimized performance criteria of assay functionality even after exposure to temperatures between about −80° C. and 70° C. Advantageously, the present reagent solutions can be conveniently stored, without refrigeration, at temperatures of about 10° C. or 15° C. up to about 30° C. or 50° C. —preferably at normal, ambient temperatures of about 20° C. up to about 27° C. as found in laboratories—over long time periods (i.e., at least ˜6-12 months) without experiencing either compositional or functional degradation, which compromises or changes the data quality or integrity as generated with the use of microarrays. Indeed, empirical results from hybridization suggest that kit solutions stored or heated, even briefly, at temperatures elevated above ambient room temperature appears to promote a desirable intensity of hybridization signal in an assay. A kit having all components stored in individual reagent containers at a common temperature (room temperature or higher) is currently not available. Moreover, these qualities can help laboratories decrease their array failure rates by as much as 10 percentage points and reduce their costs per experiment by as much as ˜40%.

According to the invention, the reagent system for gene analysis or expression assays using a nucleic acid microarray comprises:
a) a probe spotting solution containing either ethylene glycol or dimethyl-sulfoxide (DMSO) for reformulating nucleic acid probes;
b) a pre-hybridization solution containing a blocker reagent to reduce non-specific binding of targets to substrate surface or probes; and
c) a hybridization solution comprising a water soluble protein, formamide, optionally with either a surfactant or dextran sulfate, or both, and various nucleic acid blockers.

The reagent system may further include a wash reagent A comprising a buffered solution containing a citrate salt, and/or a wash reagent B comprising a buffered solution containing lauryl sulfate salt. Optionally, the reagent system may also contain a target-labeling buffer composition containing random oligonucleotide hexamers and/or primers, such as oligonucleotide dT primers, for use with total RNA, instead of mRNA, in a RNAse- and DNAse-free aqueous medium; or optionally, a background-reducing solution containing a borohydride salt to eliminate any non-bound nucleic acid, scrub printed nucleic acid of fluorescent contaminants, or reduce any oxidized amines on a substrate surface, which can arise from improper or long-term storage of the substrates, or their exposure to air contaminants. The prehybridization solution serves to further eliminate any non-bound nucleic acid and prevent smearing of nucleic acid across the substrate surface.

Each of the foregoing reagent solutions corresponds to a step in an assay protocol. The reagent system is designed to optimize the performance of microarray-based biological assays in terms of their sensitivity, dynamic range, and reproducibility, particular on γ-aminopropylsilane-(GAPS)-coated slide substrates. The system comprises several parts. First, the system may include a printing kit for depositing biological molecules onto an array substrate. Hence, the printing kit includes a nucleic acid spotting solution, also known as an ink, and a solid or semisolid, two or three-dimensional substrate having a functionalized surface. Second, the system contains a reaction kit. According to an embodiment, the reaction kit may incorporate a background reducing solution, a pre-hydridization solution for nucleic acids, a hybridization solution, and wash solutions. Both printing kit and reaction kit solutions are compositionally and functionally stable as characterized above, even though respective solutions in each kit may include individual reagent components that conventionally should not be stored together, let alone at a temperature between about 10° C. to about 55° C. for prolonged periods, such as at least over a week, or preferably over a month (e.g., ˜45 days). The individual parts of the present system may be employed separately, but preferably they are used together for best results. Specific embodiments of the system may contain variable elements and combinations of the aforementioned solutions.

The pre-soak or background reducing solution contains a reducing agent, (e.g., borohydride, BH₄⁻) at a concentration ranging from about 0.1 mg/ml to about 25 mg/ml, in a buffer solution of 4.5-10 pH.

The pre-hybridization solution contains a water soluble protein (e.g., albumin or casein) at a concentration in the range of about 0.1% wt. to about 5% or 7% wt. in aqueous medium. The water soluble protein is used to block nonspecific binding of nucleic acid to the reactive substrate surface. For cDNA microarray, specifically, the pre-hybridization solution may further contain formamide at a concentration of about 20-75%, preferably about 40-60% per volume. Additionally, a surfactant or detergent, such as sodium dodecyl sulfate (SDS) (also called sodium lauryl sulfate), is included at least about 0.01 -4%, preferably 0.1-1%. Also included are nucleic acid blockers, such as poly-A, COT-1 DNA, herring sperm DNA, or calf thymus DNA to name a few, at concentrations of about 1-500 ng/μl.

The hybridization solution contains a water soluble protein, formamide, nucleic acid blockers, and a high-molecular weight polymer, optionally with a surfactant. The high-molecular weight polymer functions as a volume excluder to increase the hybridization kinetics at the substrate surface by concentrating target nucleic acids toward the microarray surface. The volume excluder is present at a concentration of about 0.1% to about 10%; particular preferred concentrations depend on the desired specific assay protocol parameters.

In another aspect, the present invention pertains to a protocol or method for performing biological assays using the kit and stable reagent solutions described herein. The protocol comprises at least three major parts, namely, performing a pre-hybridization blocking or wash, hybridization, and a post-hybridization wash. Each of these parts may be further divided into several specific steps. The pre-hybridization wash may include the steps of: a) preheating a volume of either a pre-hybridization solution and/or pre-soak solution to a temperature higher than ambient room temperature (e.g., ˜30-50° C.), preferably, for about at least 25-30 minutes prior to b) treating or exposing a number of microarray substrates with the respective heated pre-hybridization and/or pre-soak solution within a container at a temperature higher than room temperature (e.g., ˜42° C.); afterwards, c) removing the microarray substrates and either rinsing or incubating the substrates with a washing solution; and d) drying the substrates under a purified gas stream (e.g., nitrogen) or by centrifuge spinning. The incubating step using washing solution may be repeated one or more, preferably, two times. One may also wash each substrate with deionized water (e.g., ultra-pure water of ˜10-18.5 MegaOhm (MΩ), preferably ˜17-18.2 MΩ, resistivity); but, doing such is not recommended for DNA spotted assays. Hybridization may include the steps of: a) dissolving a predetermined amount of fluorescently-labeled nucleic acid in a volume of a hybridization solution; b) incubating the nucleic-acid solution at a temperature of about 95° C.±3° C.; c) collecting the nucleic-acid condensation, and allow the solution to cool to room temperature; d) applying a target-containing solution and covering the microarray; e) placing a prepared microarray in a hybridization chamber; f) applying a target-containing solution and covering the microarray; and g) incubating the microarray at about 42° C.±3° C. for about three to 24 hours, preferably between about 12 to 16 or 18 hours. The post-hybridization wash may include the steps of: prepare a container with pre-warmed washing solution at a temperature of about 35° C. to about 50° C., preferably about 42° C.; remove the microarrays from the hybridization chamber and either wash or incubate the microarrays with post-hybridization washing solutions for about 5 minutes ±2 minutes; and dry the microarrays either under a purified gas stream or by centrifugation. One should not allow the arrays to dry out between washes, as this tends to result in high and uneven background signal. Multiple containers may be used to perform the washes in the most efficient manner.

Additional features and advantages of the present invention will be revealed in the following detailed description. Both the foregoing summary and the following detailed description and examples are merely representative of the invention, and are intended to provide an overview for understanding the invention as claimed.

### DESCRIPTION OF THE FIGURES

FIG. 1 shows a schematic flow-chart for a protocol using the present invention, in which a reagent solution is provided for each corresponding step of the protocol. The assembly of reagent solutions is referred to as a reagent system.

FIG. 2 compares a series of fluorescence images for nucleic acid microarrays on different substrates. Column A represents an array formed on a γ-aminopropylsilane-(GAPS)-coated slide, and Columns B-D, each represents an array printed on three other commercially available amine-presenting substrates. An assay is performed on each microarray. Column A presents images obtained at three major points of the assay protocol according to the present invention. Columns B-D represents corresponding images for assays according to the respective vendor's recommended protocol.

FIG. 3 is a demonstration of the high-sensitivity expression profiling, according to the present invention, with total RNA on a human 2K cancer array. Fluorescent image A represents the microarray after hybridization with a specific amount of fluorescently labeled cDNA generated initially from ˜5.0 μg RNA, which was isolated from untreated MCF breast cancer cells, following self-self hybridization. Fluorescent image B presents another similar microarray after hybridization with a specific amount of fluorescently labeled cDNA generated initially from ˜5.0 μg RNA, which was isolated from vitamin D-treated MCF breast cancer cells. Labeled cDNA material from treated cells was read using the Cy5-channel relative to labeled cDNA from untreated cells, which was read using the Cy3-channel. The difference in expression profiles shown in the images A and B, are presented in graphs C and D, respectively. The results confirm that treatment of the cancer cells with vitamin D lead to the up-regulation of vitamin D-24 hydroxylase gene.

FIG. 4 is a demonstration of the high-sensitivity gene-copy detection, according to the present invention, using a bacteria gene spiking experiment on a human 10K array. Different amounts (1 μg, 0.5 μg, 0.25 μg, 0.125 μg and 0.075 μg) of in vitro transcripts of bacteria genes (yabQ, yacK, ybaS, and ybbR) labeled with Cy5 dye are spiked into a background of Cy3-labeled human brain and Cy5-labeled human testis cDNA generated from with ˜4-5 μg of total RNA. A specific amount of labeled cDNA is added for hybridization; typically added based on the size of the glass coverslip used for hybridization. The amount of labeled cDNA corresponds to a pmol value as calculated from optical density measures of the labeled cDNA. (See FIGS. 14A-F for the calculations and procedures used.) For example, 36-50 pmol of labeled cDNA is used for hybridization when using a 24×60 mm glass coverslip. The key to reproducibility, consistency, low coefficients of variation, no non-specific hybridization, and background control is in the uniform addition of labeled cDNA per hybridization. The quality and consistency of the labeled cDNA material added for hybridization must be critically controlled. Fluorescent image A presents one subgrid of the 10K array after hybridization. The graph B presents a plot of Cy5 signal-to background ratio versus gene copy number per cell. Results indicate that the sensitivity of the assays performed using the present reagent kits is better than one copy in 0.5×10⁶ cells, which is about 5-10 folder better than leading competitive kits.

FIG. 5A is a demonstration of high reproducibility of a gene expression profile, according to the present invention, using a human 2K cancer array. The graph shows the ratio of Cy5/Cy3 for RNA from D3-treated MCF cells between two slides, each having duplicate subarrays. The median variance of the ratio is about 5-6% between the slides or between subarrays on the same slide. FIG. 5B is a graph of CV for 4K arrays under five assay conditions, using three slides per condition.

FIG. 6 shows a graph of the evaporation rate of the spotting or printing ink according to the present invention, in comparison with six other commonly used spotting solutions. The spotting solution according to the present invention shows considerably lower evaporation rate. A lower evaporation rate allows researchers the flexibility to perform longer and more consistent printing runs for microarry fabrication. Moreover, because of less evaporation, more slides can be printed per set of nucleic acid probes, thus decreasing the overall cost of each array printed. The spotting solution is critical to optimum array performance and must be specifically matched to the substrate being used for printing. The spotting solution described in this system was optimized or selected for the following attributes: spot uniformity, spot size, spot consistency, optimal DNA retention, optimum interaction with the surface chemistry of the slide (e.g., specifically GAPS), nuclease inhibition characteristics, hygroscopic nature for limited evaporation, shelf life for printing, chemical stability and compatibility with nucleic acids, low florescence characteristics, and compatibility with quill type or solid pin printing devices.

FIG. 7 is a demonstration of the efficiency and reproducibility of the present invention for labeling target RNA by use of random primers.

FIG. 8 is a demonstration, according to the present invention, of the effective reduction of auto-fluorescence background of the microarray and its substrate surface using a reducing reagent such as borohydride. The three images A-C, highlight the dramatic reduction in background after treatment with the reducing reagent. The graph D summarizes the statistical results of the present solution relative to five comparative microarrays on different substrates surfaces using products from other commercial vendors.

FIGS. 9A and 9B, respectively, are graphs showing the net signal ofCy3 and Cy5 for a human 6K cancer microarray after self-self hybridization with human brain RNA, with or without treatment with a background reducing solution according to the present invention. Treatment with the reducing solution lowers the total background baseline, as it enhances the sensitivity and dynamic range of the expression data. The reducing solution, as described, serves two main functions. One, upon addition of for instance, NaBH₄ tablets or powder to the pre-soak buffer solution, the solution begins to effervesce, generating a mechanical scrubbing action to the printed nucleic acid material, which reduces any background associated with the printed material. Any florescence background associated with printed content is a potential artifact from upstream production or purification procedures that leave residual salts or chemicals. These florescent materials typically are not removed through pre-hybridization or hybridization processes, which leads to high standard deviations in signals generated from printed spots. Second, NaBH₄ acts as a chemical reducing reagent to reduce any oxidized amines on the surface of the slide, which may have been generated over long term storage conditions, improper storage, improper post printing conditions, or exposure to air contaminants that cause oxidation.

FIGS. 10A and 10B are graphs that show the signal-to-background ratio of 4K genes on a microarray after self-self hybridization with a specific amount of labeled cDNA as generated from ˜4.5 μg of total testis RNA, respectively, in the presence and absence of dextran sulfate (6%) in the hybridization buffer solution. The addition of dextran sulfate (DS) can improve signal-to background ratios because it not only increase the viscosity of solution but also increases the local concentration of target nucleic acids near the probes on the surface.

FIGS. 11A and 11B are images of two identical microarrays that have undergone hybridization, respectively, with 25% formamide and with 50% formamide in hybridization buffer. The advantage of using a hybridization solution containing 50% formamide over one with 25% formamide is a gain in specificity. Lower formamide leads to non-specific binding, higher signal and false expression levels.

FIGS. 11C-11F are graphs and charts quantifying the data from FIGS. 11A and 11B, respectively.

FIGS. 12A and 12B show the relative accelerated stability of hybridization studies carried out using a reagent kit according to an embodiment of the present invention. The y-axis in FIG. 12A presents data for the signal to background noise ratio, and in FIG. 12B presents data for net signal fluorescence.

FIGS. 13A and 13B show the relative accelerated stability of hybridization studies using a reagent kit according to another embodiment of the present invention. The y-axis in FIG. 13A presents data for the signal to background noise ratio, and in FIG. 13B presents data for net signal fluorescence.

FIGS. 14A-14F show the calculations and procedures for accurate characterization of labeled cDNA and accurate addition into hybridization reactions, which provides premium control in terms of sensitivity, reproducibility, consistency, low coefficient of variation, no non-specific hybridization signals, and background signal.

### DETAILED DESCRIPTION OF THE INVENTION

### Section I—Definitions

Before describing the present invention in detail, it is to be understood that the terminology used herein is for the purpose of describing particular embodiments only, and is not intended to be limiting. As used in this specification and the appended claims, the singular forms “a,” “an,” and “the” include plural referents unless the context clearly dictates otherwise. All technical and scientific terms used herein have the usual meaning conventionally understood by persons skilled in the art to which this invention pertains, unless context defines otherwise.

As used herein, the term “biological molecule” refers to any kind of nucleic acid entity, including, such as, oligonucleotides, DNA, RNA, peptide nucleic acid (PNA). The nucleic acid can take the form of either double stranded or single stranded molecules. When a single stranded molecule is used, the nucleic acid can either have a secondary structure or not.

As used herein, the term “biospot” or “microspot” refers to a discrete or defined area, locus, or spot on the surface of a substrate, containing a deposit of biological or chemical material.

As used herein, the term “probe” refers to a biological molecule, which according to the nomenclature recommended by B. Phimister (Nature Genetics 1999, 21 supplement, pp. 1-60.), is immobilized to a substrate surface. Preferably, probes are arranged in a spatially addressable manner to form an array of microspots. When the array is exposed to a sample of interest, molecules in the sample selectively and specifically binds to their binding partners (i.e., probes). The binding of a “target” to the probes occurs to an extent determined by the concentration of that “target” molecule and its affinity for a particular probe.

As used herein, the term “substrate” or “substrate surface” as used herein refers to a solid or semi-solid, or porous material (e.g., micro- or nano-scale pores), which can form a stable support. The substrate surface can be selected from a variety of materials.

As used herein, the term “complement” or “complementary” refers to the reciprocal or counterpart moiety of a molecule to another. For instance, complementary nucleic acid sequences, in which nucleotides on opposite strands that would normally base pair with each other mostly according to Watson-Crick-base pair (A/T, G/C, C/G, T/A) correspondence.

### Section II—Description

In heterogeneous assays, nucleic acid arrays may comprise a number of individual sequence species (e.g., cDNA, ssDNA,genomic DNA, or oligonucleotide) immobilized or tethered to the surface of a solid support in a regular pattern, each species in a different, distinct area, so that the location of each sequence is known. The performance of nucleic acid microarrays are related to several factors. One is the deposition or printing quality of probe microspots. Another is the hybridization efficiency or stringency, and specificity of the interaction between target sequences and probes, which are influenced by reagents employed and/or environmental conditions of the assay. Still, another factor is the level of background signal due to either the auto-fluorescence of the substrate or probe microspot, and/or non-specific binding of the labeled targets to the substrate.

Microarray quality is highly dependent on the quality and integrity of both the substrate surface and the probe microspots printed on the substrate surface. Arrays printed, for instance, on a coated glass surface of poor quality are likely to produce spots of varying size, shape, and nucleic acid content. The presence of scratches, haze, and contaminating particulates on the slide surface also cause deformation of the arrays as well as high background fluorescence. These problems lead to loss in sensitivity and generally poor results. The composition of the spotting solution or inks used can affect significantly the quality of the deposited probe microspots, including morphology, reproducibility and consistency. An ideal spotting or printing solution should satisfy certain parameters. Generally, the printing solution should have a viscosity that is sufficiently high to minimize loss of the solution from evaporation during the fabrication process, and to permit a printing pin or other contact device to pick-up and transfer a reproducible amount of the solution onto a prepared substrate. Further, the printing solution should also maximize the immobilization of probes with the substrate surface after deposition, and avoid the formation of nucleic acid sequence secondary structures to allow maximal interaction of probes with targets during hybridization. An excessive loss of probe DNA sequences can lead to a low fluorescent-signal-to noise ratio and uncertain or erroneous results.

The quality and reliability of microarray results also depends on the quality and consistency of the reagents used to process the arrays. Hybridization efficiency or stringency, and specificity of the interaction between target sequences and probes can significantly influence array performance. These parameters can be controlled through optimization of reagent compositions throughout the assay protocol and/or environmental conditions.

High background and low signal to noise performance are leading causes of poor results and array failures for laboratories. The present reagent systems can generate high signal-to-noise ratios, which promotes higher confidence level for measuring slight changes in low-expressing genes. For enhanced signal-to-noise performance according to the present invention, one can reduce background signal by two to three-fold relative to average industry levels, especially if one pre-soaks an array in solution. The high reproducibility, low background and high reliability of the present reagent systems for microarrays can help laboratories decrease their array failure rates by up to about 50% relative to the industry average failure rate, and reduce their costs by as much as nearly 50% per microarray experiment.

Hence, the present invention provides an optimized reagent system for printing, assaying, and processing microarrays. In general, the system comprises: a printing kit and a hybridization kit. The printing kit has a nucleic acid spotting solution or ink. The hybridization kit includes a nucleic acid pre-hybridization solution, a nucleic acid hybridization solution, wash solutions, and preferably a background reducing solution. The hybridization kit may further include an optimized first, second, and/or third wash reagents. The kits also incorporate optimized protocols for labeling and purifying of fluorescent labeled cDNA, and optimized protocols for addition of labeled cDNA for hybridization, followed by recommended substrate drying procedures. The formulations of reagents in each kits combined with the techniques employed according to the present invention affords a synergistic advantage, which produces better functionality and results than one would conventionally expect.

The system is embodied in two general families of solutions, one formulated preferably for cDNA (PCR product) microarray applications, and another more general or “universal” system optimized preferably for assays using microarrays of both cDNAs and long-length oligonucleotides (e.g., ≧30 nucleotides, preferably ≧49-55 nucleotides). The cDNA version offers a commercialized version of the current recommended protocols but with a new, improved spotting solution. While both families of the system can out-perform industry standards, some attributes of reproducibility, sensitivity and background are improved even further in the “universal” system relative to the cDNA version. The cDNA version does not contain the pre-soak treatment, which is highly recommended, and does not contain hybridization enhancers such as the dextran sulfate used for volume exclusion. Another difference between the two families is that the universal system offers increased performance at a lower statistical coefficient of variation (CV), higher sensitivity, and the versatility of the ability to use both long-length oligonucleotides and cDNA content with buffers and protocol described herein. This difference is reflective of the protocols used for labeling and purification of the labeled cDNA as well as in the wash conditions used after hybridization; stringency control.

Relative to other reagent solutions available commercially in today's market, the present reagent-systems platform exhibits enhanced attributes of reproducibility, low background, high signal-to-noise ratios and consistent spot morphology, which act together to deliver high quality data. When performing assays, using for instance γ-aminopropylsilane (GAPS)-coated slides, the present reagent systems deliver reliable, reproducible data, which results in less waste of supplies, reagents, samples and labor. Users can achieve consistent results between experiments and between separate array slides, with a deviation of about 10% or less. Reproducibility is one of the most important attributes for end users, followed closely by sensitivity, which is determined by background and signal-to-noise ratios.

According to another advantage of the present invention, we have discovered that reagent kits or solutions used for biological assays can remain stable, retaining their functional performance, even when stored together at temperatures between about 10° C. to about 50° C., for extended periods of about six months or longer. Preferably the reagents are stored between about 14° C. to about 45° C., more preferably between about 17° C. to about 35° C., or even more preferably at about ambient room temperatures (e.g., ˜20-28° C.). Synergy for a combination of different components targeted for the use in each step of an assay is achieved all in one solution. Different constituent components of an individual reagent solution may be mixed together ahead of time, and each set of solution components can be stored together in a single container over extended periods of time without the solution experiencing either physical degradation or loss of function. This discovery presents a phenomenon contrary to conventional understanding of reagent storage practices, which believes that different reagents either should not or could not be prepared and stored together in a pre-mixed assay solution under a single temperature or environmental condition and still be able to maintain the solution in good functional state. Current laboratory practices store reagents used for microarray assays under refrigeration at about 4° C. or below. For instance, manufacturers recommend typically that dextran sulfate and formamide be maintained at 4° C., whereas bovine serum albumin (BSA) should be stored at4° C. to −20° C. According to the invention, when reagent kits are stored at temperatures higher than conventional, one can achieve better hybridization performance, hence better overall quality of the assay. It is believed that greater solubility of components at higher temperatures may drive the reaction kinetics. Better or faster hybridization kinetics as well as optimizing steric orientation of probe molecules can lead to manifestations of enhanced signal and reproducibility. Also, note that all hybridization reagent components in the presence of labeled cDNA can be heated at 95° C. for 3-5 minutes for the purpose of eliminating secondary structure of the cDNA without any deleterious effect to the solution performance during hybridization.

Further, other beneficial features of the present invention may include more efficient and consistent array preparation. Since the printing or spotting solutions have low-evaporation, greater numbers of slides may be printed per library plating and at more consistent DNA concentrations over longer print runs. Significant adjustment of the solutions between print runs is not required. Optimized long oligonucleotide and cDNA spotting solutions provide consistent, uniform features with no crescents, doughnuts or blotches, which may be observed with other solutions. The present spotting solution has very low background fluorescence characteristics, again improving array performance. Furthermore, the spotting solution provides nuclease inhibition so that the nucleic acid content is protected from degradation during storage.

### A. Assay Protocol

In terms of the properties of arrayed nucleic acid sequence with known identity, the majority of DNA microarrays generally come in two variants: cDNA and oligonucleotide arrays; although, genomic DNA arrays are now becoming popular as well. In a cDNA microarray, cDNAs (500˜5,000 bases long) are employed as probes. Whereas in an oligonucleotide microarray, oligonucleotides (20˜80-mer oligos) or peptide nucleic acid (PNA) probes are synthesized either in situ (on-chip) or by conventional synthesis with subsequent immobilization on the substrate. Assay protocols for using these two main types of nucleic acid microarrays, however, are similar. Typically, each protocol involves multiple steps, including pre-hybridization to block the background and remove un-immobilization probe molecules, hybridization to allow the interaction of target sequences with the probe molecules, and washing to remove unbound targets as well as non-specific and weakly bound targets to non-complementary probe sequences, followed by detection.

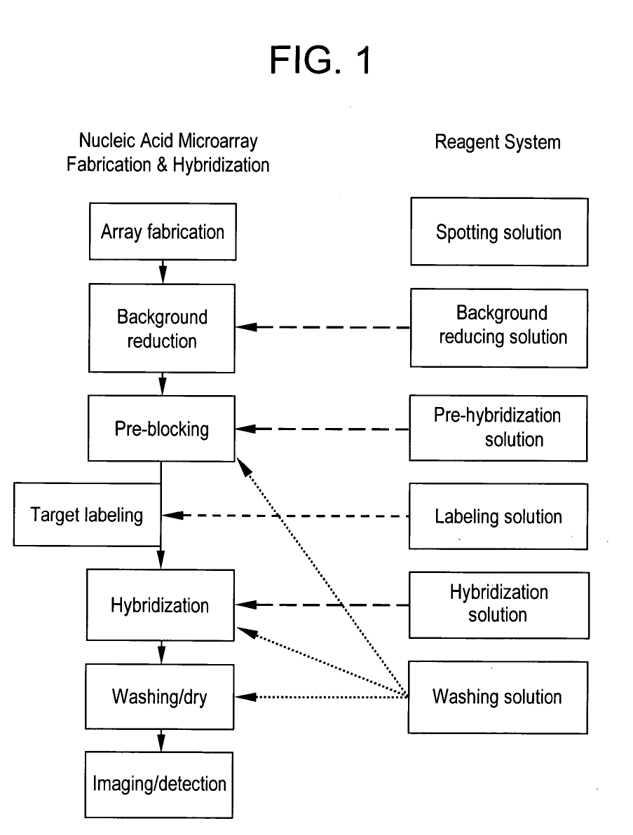

According to an embodiment, the present invention standardizes and streamlines the assay protocol for nucleic acid microarrays. The assay protocols, as depicted schematically in FIG. 1, comprise the major parts, namely, a pre-soak,a pre-hybridization blocking or wash, labeling and purification, hybridization, and a post-hybridization wash. Each of these parts may be further divided into several specific steps. The pre-hybridization wash may include the steps of a) preheating a volume of either a pre-hybridization solution or pre-soak solution to a temperature higher than ambient room temperature (e.g., ˜30-50° C.), preferably, for about at least 25-30 minutes prior to b) treating or exposing a number of microarray substrates with the respective heated pre-hybridization or pre-soak solution within a container at a temperature higher than room temperature (e.g., ˜42° C.); afterwards, c) removing the microarray substrates and either rinsing both sides of each substrate with deionized water (e.g., ultra-pure water of ˜10-18.5 MegaOhm (MΩ), preferably ˜17-18.2 MΩ, resistivity) or incubating the substrates with a washing solution; and d) drying the substrates under a purified gas stream (e.g., nitrogen) or by centrifuge spinning. The incubating step using washing solution may be repeated one or more, preferably, two times. Hybridization may include the steps of: a) dissolving a predetermined amount of fluorescently-labeled nucleic acid in a volume of a hybridization solution; b) incubating the nucleic-acid solution at a temperature of about 95° C.±3° C.; c) collecting the nucleic-acid condensation, and allowing the solution to cool to room temperature; d) placing a prepared microarray in a hybridization chamber; e) applying a target-containing solution and covering the microarray; and f) incubating the microarray at about 42° C.±3° C. The post-hybridization wash may include the steps of: preparing a container with pre-warmed washing solution at a temperature of about 35° C. to about 50° C., preferably about 42° C.; removing the microarrays from the hybridization chamber and either wash or incubate the microarrays with post-hybridization washing solutions for about 5 minutes ±2 minutes; and dry the microarrays either under a purified gas stream or by centrifugation. One should not allow the arrays to dry out between washes, as this tends to result in relatively high and uneven background signal. Multiple containers may be used to perform the washes in the most efficient manner.

### B. Reagent System

The present invention provides, in part, an assembly or reagent system for performing biological assays using nucleic acid microarrays. The system involves the use of at least one or more reagent kits or compositions adapted for various steps of an assay process. Each solution contains one or more components in a single medium.

According to the invention, the reagent system for gene analysis or expression assays using a nucleic acid microarray comprises:
a) a probe spotting solution containing either ethylene glycol or dimethyl-sulfoxide (DMSO) for reformulating nucleic acid probes;
b) a pre-hybridization solution containing a blocker reagent to reduce non-specific binding of targets to substrate surface or probes; and
e) a hybridization solution comprising a water soluble protein, formamide, optionally with either a surfactant or dextran sulfate, or both.

The reagent system can include solutions for washing, in particular a “wash reagent A” comprising a buffered solution containing a citrate salt, and/or a “wash reagent B” comprising a buffered solution containing a lauryl sulfate salt. One may also include additional components such as a target-labeling buffer composition containing random oligonucleotide primers, such as hexamers or oligonucleotide dT primers, and optimized nucleotide formulations (labeled versus unlabeled, for mRNA versus total RNA species), optimized reverse transcriptase concentrations designed to interact with total RNA in place of mRNA, in a RNAse- and DNAse-free aqueous medium. Also included is a solution containing a borohydride salt used to reduce background autofluorescence. Furthermore, the reagent system and method of use includes protocols for target-label purification, optical density measurements of labeled cDNA, and final quantification for additions of a pmol amount of labeled target per hybridization.

The foregoing reagent systems are specifically tuned to and optimized for use with the microarrays of both cDNAs and oligonucleotides such that researchers may achieve the highest possible level of performance, standardization, and technical control throughout the microarray processes.

### C. Probe Spotting Solution

A number of printing technologies have become amenable to production-scale fabrication of nucleic acid microarrays. The most popular ones are contact pin printing and non-contact ink-jet printing.

Contact pin arrayers generally deliver sub-nanoliter volumes of nucleic acid probe solution directly to a surface using a tiny pin with or without capillary slot (“quill pin” versus “solid pin”, respectively). The use of quill-pin printers is more suitable for large-scale production of nucleic acid microarrays, because one sample pickup can produce tens or even hundreds of reproducible and consistent microspots in a single slide or among multiple slides.

On the other hand, the ink-jet arrayers, for example, involve precise drop deposition using a thermal-driven bubble inkjet device, or using a piezoelectric pump, such as described in U.S. Pat. No. 5,474,796. In the latter one, a piezoelectric pump delivers minute volumes of liquid to a substrate surface. The pump design is very similar to the pumps used in ink jet printing. This pico-pump is capable of delivering a droplet of 50 micron diameter (˜65 picoliter) at up to 3000 Hz and can accurately hit a 250 micron target. The pump unit remains stationary while droplets are fired downward at a moving array plate. When energized, a microdroplet is ejected from the pump and deposited onto a substrate surface to form a microarray. Generally, this type of printer is less restricted to surface structure.

No matter which printing technologies used for array fabrication, considerable optimization is required to prepare high-quality microarrays. For instance, the nucleic acids should be buffered in a solution that leads to optimal printing reproducibility with desired spot morphology. The preparation of nucleic acid microarrays could be significantly slow when a greater number of elements are arrayed or numerous microarrays are produced. Therefore the loss-of the probe solution during the printing process, due to the evaporation, should be minimized. For large volume manufacture of printed microarrays, evaporation of the spotting solution during the deposition process is a major limiting factor. Undesired evaporation results in progressively concentrated levels of organic components, thereby leading to a constantly changing ink composition and inconsistent printing quality.

Some special concerns relate to the printing technology employed. For example, if contact printing technology is used, pin-contact time and the force with which a pin strikes the array substrate should be modified, depending on the wetting properties and nature of surface chemistry as well as surface topology. In addition, a pin cleaning protocol may also be included to avoid cross-contamination between samples. Furthermore, the wettability of components such as printing pins and coated slide surface can be affected by the composition of the printing or spotting solution. In the manufacture of arrays, it is desirable for a nucleic-acid ink to be able to wet thoroughly contact printing pins and to allow probe materials transfer reproducibly from the pins to a functionalized surface of a substrate. In other words, the ink should adhere to the pins and adsorb to the surface in large amounts. Thus, one must make careful determination of pH and other parameters, including salt and organic solvent concentrations.

The present invention addresses specifically the foregoing problems. According to an embodiment, the printing ink or spotting solution comprises: about30% to about 94% or 96% by volume of an organic solution comprising dimethylsulfoxide (DMSO), ethylene glycol (EG), formamide, or a combination thereof; a buffer with a pH value of about 3.5-9.5, preferably about 6 to about 8.5, more preferably about 6.5 to about 7.5; water; and optionally predetermined nucleic acid sequences. Preferably the composition comprises about 40% to about 80% or 87% by volume of DMSO, EG, formamide, or a combination thereof. The buffer can be made from a solution that may include acetate, citrate, citrate-phosphate, maleate, or succinate. The nucleic acid denatures to provide for more favorable hybridization. When the buffer includes citrate, the pH value is about 3.5 to about 7.5, preferably about 4 to about 6.5. When the buffer includes citrate-phosphate, the pH value is about 6.0 to about 9, preferably about 7 to about 8.5. When the buffer includes succinate, the pH value is about 3.5 to about 7, preferably about 4 to about 6.5. When the buffer includes maleate, the pH value is about 5 to about 8.5. When the composition contains either ethylene glycol or formamide, the maleate buffer is at a pH value of about 5-5.5. When the composition contains DMSO, the maleate buffer is at a pH value of ˜8 to 8.5. The nucleic acid is at a concentration ranging from about 0.01 mg/ml to about 0.5 mg/ml. The nucleic acid can be a double-stranded DNA, genomic DNA, cDNA, RNA, or an oligonucleotide. Other details for similar nucleic acid ink compositions are discussed in U.S. patent application Ser. No. 10/244,898, by S. Pal, the content of which is incorporated herein by reference.

Alternatively, the spotting solution has a composition comprising: a mixed organic solution of about 1% to about 55% by volume of ethylene glycol (EG) or formamide either individually, together in combination, or with DMSO; a buffer with a pH value of about 3.5-9.5; water; and optionally nucleic acid. The ink composition enables long-term storage and preserves integrity of nucleic acid without instability by precipitation or aggregation of said nucleic acid. In other words, the composition enables prolonged storage and printing over at least 15 days, preferably of at least about six months. The ink can be stored up to about 12 months without significant degradation or appearance of artifacts in assays.

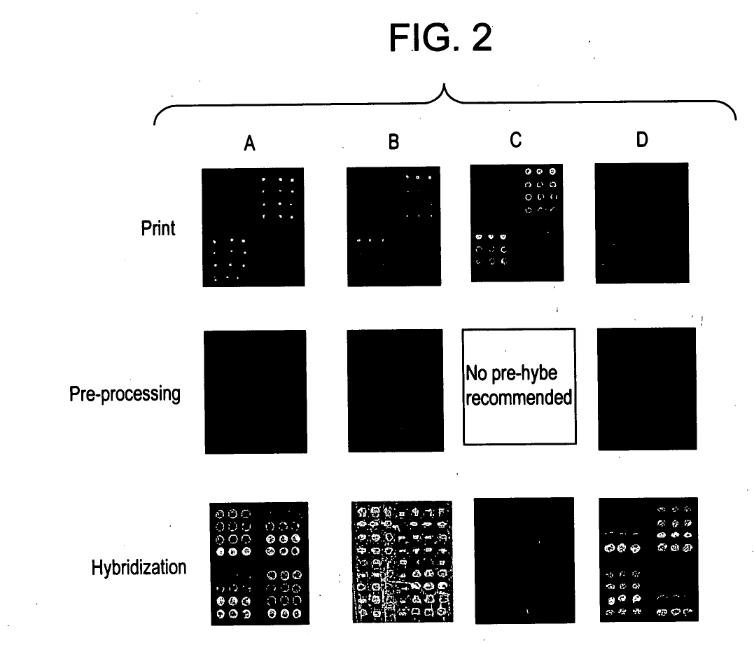

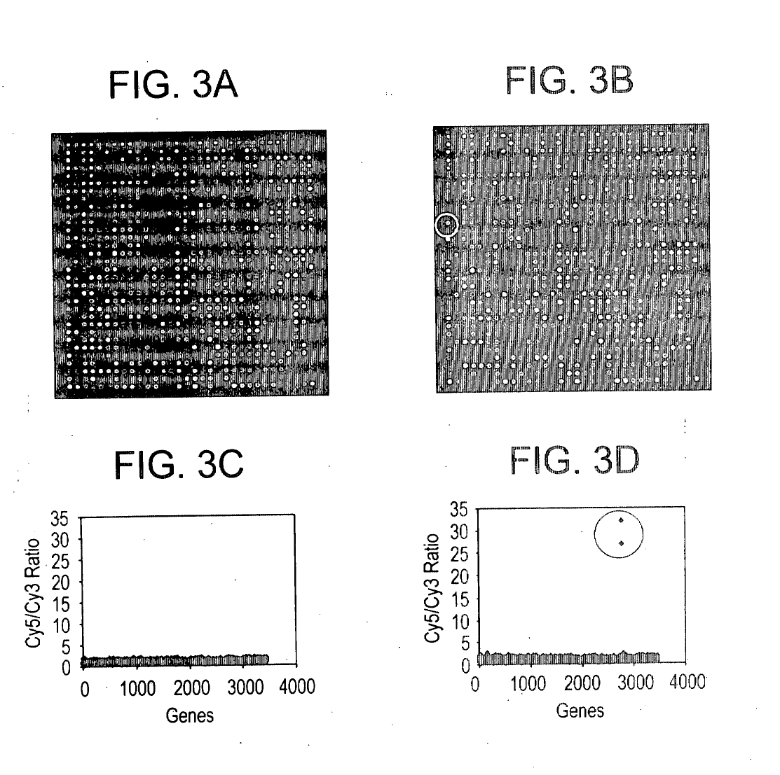

The use of the spotting solutions results in enhanced printing quality and hybridization performance. As shown in FIG. 2, the spotting solution according to the present invention is used to fabricate arrays of cDNAs on GAPS-coated slides of glass (e.g., Corning® UltraGAPS™). The resulting arrays exhibit uniform spot size and consistent morphology throughout the assays, and low auto-fluorescence background before and after hybridization (2A). Moreover, the binding of the targets to their corresponding probe microspots shows high specificity and reproducibility with high assay sensitivity (high signal-to-noise ratio). In contrast, assays performed using three other spotting solutions from different commercial vendors, following their respective, recommended protocols, results in high failure rate in array fabrication, due to either high auto-fluorescence signals from the microspots and low assay sensitivity (2B), low binding signals of targets to the probe microarrays (2C), or relatively high auto-fluorescence signals from the microspots and undesired spot morphology (2D).

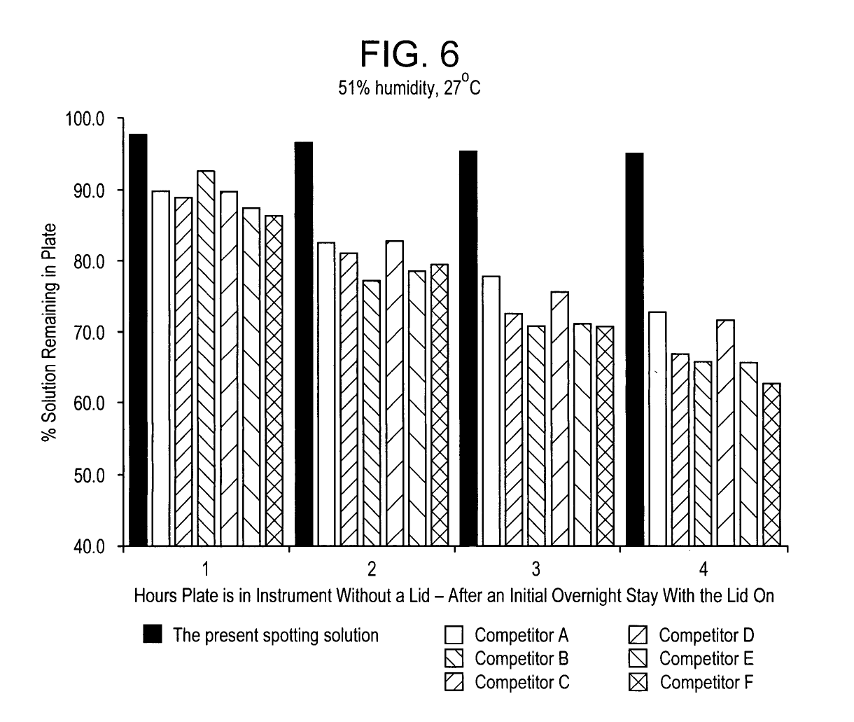

In addition, the printing solutions according to the present invention show the lowest evaporation rates among these spotting solutions tested, resulting in greater stability of the biological content and lower print failure rates, relative to other commonly used printing solutions. The spotting solutions are hygroscopic and demonstrate about 5% evaporation after about 4 hours without a cover lid, as shown in FIG. 6. The losses and inconsistency normally experienced during extended printing is not an issue with the present inventive system, and the number of slides printed per library plating can be greater than that which is achievable with comparable, commercial formulations available currently.

Table 1 summarizes, in terms of certain parameters that are considered when benchmarking against industry averages, the relative, improved performance attributes of the present reagent system, for both universal and cDNA families.

TABLE 1
Attributes of probe spotting solution on DNA microarrays.

| | | |
|---|---|---|
| cDNA and/or | | |
| oligonucleotides | cDNA | Current Industry |

Spotting Solution Attributes

| | | | |
|---|---|---|---|
| Evaporation Rate | 5% | 5% | 35% |

Hybridization Attributes

| | | | |
|---|---|---|---|
| Interslide Deviations | ˜5% | ˜10% | ˜15% |
| Background Signal | 125 RFUs | 200 RFUs | 600-700 RFUs |
| Signal to Noise Ratio | 3+ | 2+ | 1+ |
| (relative levels of | | | |
| improvement) | | | |

### D. Solution for Labeling Target Nucleic Acid

In general, for nucleic acid microarrays, RNAs arte labeled with fluorescence tagged nucleotides using a reverse transcriptase (RT) enzyme and optimized reaction components, such as primers, nucleotides and reaction buffer with metal ion salts, such as magnesium chloride. These labeled cDNAs, which are used as targets during hybridization, contain poly-A regions of different lengths. Commonly, poly-dT primers are used for primer extension to label total eukaryotic or messenger RNA samples. Since the primer extension with poly-dT or anchored poly-dT starts reverse transcription (RT) from the 3′ end of mRNA exclusively, the labeling near the 5′ end of RNA is not as efficient as near the 3′ end as a consequence of either the secondary structure of RNA, the length of the mRNA, or steric hindrance of fluorescent dyes and due to early termination of the transcription process. This could be problematic, especially for DNA oligonucleotide arrays. For a given gene the specific hybridization signal could be lower for targets located near its 5′ end than for targets near its 3′ end, which in turn could affect the ratios. In addition, the labeling efficiency and frequency can dramatically affect the assay sensitivity. The optimal frequency of incorporation (FOI=# of dye-labeled nucleotides per 1000 nucleotides) of a target sequence is preferably between 10 and 50 dye-labeled (Cy3/Cy5) nucleotides per 1000 nucleotides. Lower incorporation will affect the assay sensitivity. An FOI greater than 50 dye-labeled nucleotides per 1000 is also sub-optimal due to low hybridization efficiencies believed to be due to steric hindrance from the cyanine dye molecules.

According to the present invention, a target-labeling buffer composition is provided to improve the efficiency and consistency of target nucleic acid labeling. In one embodiment, the composition may contain a random selection of oligonucleotides, such as hexamers (6mers) or 9mers, as primers. Preferably, this composition is used for labeling mRNA. Alternatively, the composition may contain an oligonucleotide dT primer. Optionally, the two types of primers are combined in the same labeling solution. This combined composition, preferably, is employed for labeling total RNA. The labeling solution is buffered and does not contain RNAse or DNAse.

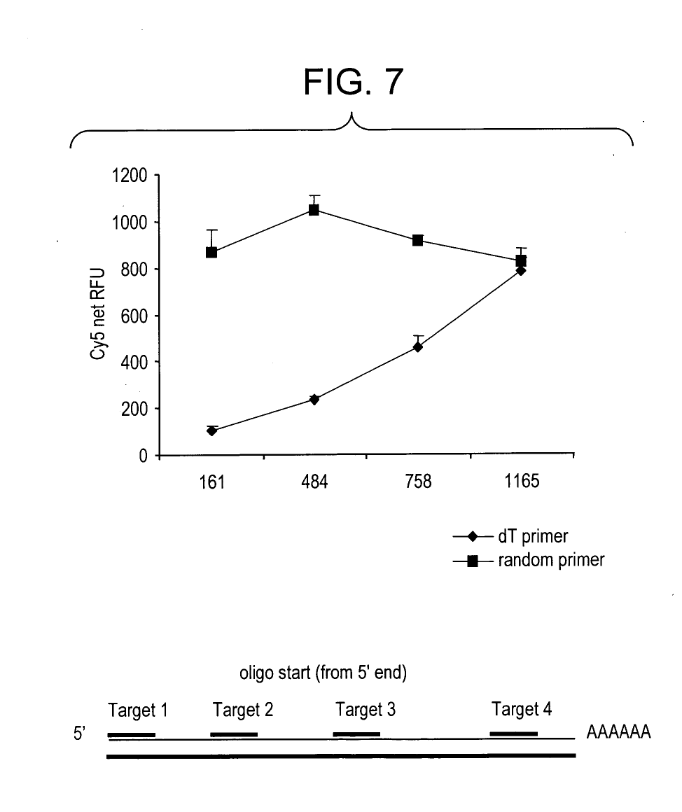

The advantage of using a random oligonucleotides as primers is demonstrated in FIG. 7. In FIG. 7, a set of DNA targets for B. subtilis gene is deposited on an array. A sample of 1.2 kb B. subtilis RNA (with an engineered poly-A tail) is produced using in vitro transcription. For the set of tiling oligonucleotides, 4 oligonucleotides (60mers) are synthesized to cover the whole length of the RNA molecule. Each oligonucleotide was about 300-400 nucleotides apart. These oligonucleotides are printed on GAPS-coated slides as probes. RNA is labeled by reverse transcription with either poly-dT primer or semi-random primers. The hybridization results show that both the Cy3 and Cy5 signal with poly-dT labeled probe is similar to random primer labeled probe near the 3′ end of RNA, indicating both primers work with similar efficiency. The hybridization signal, however, dropped significantly for the targets near the 5′-end with poly-dT primer labeled probe, as depicted in FIG. 7. This reflects the reduced transcription efficiency of the 5′ end compared to the 3′ end and reveals the advantage of using random primer over poly-dT primers during reverse transcription. It is believed that according to the present invention, the primer concentrations and nucleotide concentrations (labeled verses non-labeled) are optimized to provide the best FOI as well as the best size distribution of labeled products for hybridization of oligonucleotide and cDNA arrays.

### E. Background-Reducing Solution

A background reducing solution containing a reducing agent, such as NaBH₄ is part of the present reagent system to address background auto-fluorescence, such as described in greater detail in U.S. patent application Ser. No. 09/925,808, incorporated herein by reference.

Fluorescence imaging technologies are the primary detection methodologies used for microarray technology. The detection sensitivity could be severely compromised by background signals, which may originate from endogenous sample constituents/surface to which the probes are immobilized or from nonspecific hybridization of targets to the probes. Generally, the nonspecific signals referred to as “noise signals,” but not the intrinsic auto-fluorescence, can be greatly minimized or even eliminated by a high stringent hybridization and/or a high stringent wash of arrays after hybridization. The intrinsic auto-fluorescence of the arrays (both the spare surface and the microspots) not only affects the assay sensitivity (thereby affecting the accuracy and consistency of assay results, such as overestimating the gene copies), but also obscures the sensitivity of gene expression analysis to a large extent by hindering the detectability of the low-level specific fluorescent signals.

Potential sources of auto-fluorescence are multiple. Auto-fluorescence could be due to trace impurities of fluorescent molecules that typically contain single or conjugated pi-orbital bonding. In addition, during storage or printing, adsorption and oxidation of some biological or chemical contaminants, could result in the emission of fluorescence. Applicants have discovered a relatively rapid, reproducible and easily applicable method to reduce substantially auto-fluorescence of slide surfaces as well as the probe microspots. The method involves the treatment of the printed arrays on a substrate surface by employing a background-reducing solution containing reducing reagents.

According to one aspect of the invention, the reducing agent is selected from the group consisting of hydrides. Applicants have surprisingly discovered that treatment with a reducing agent such as a hydride significantly diminishes auto-fluorescence on the surface of the substrate as well as on the locations deposited on the substrate. In a preferred aspect of the invention, the reducing agent includes a borohydride, and more preferably, sodium borohydride. According to a most preferred aspect of the invention, the sodium borohydride is in a solution at a concentration ranging from 0.01% to 1% weight per unit volume. Other potential reducing agents that may be used in accordance with the invention include sodium cyanoborohydride and copper sulfate. The reducing reagent is preferably in a buffered solution containing 2×SSC at a concentration of about 300 mM sodium chloride, 30 mM sodium citrate, at a pH of about 6.3 to about 9.5.

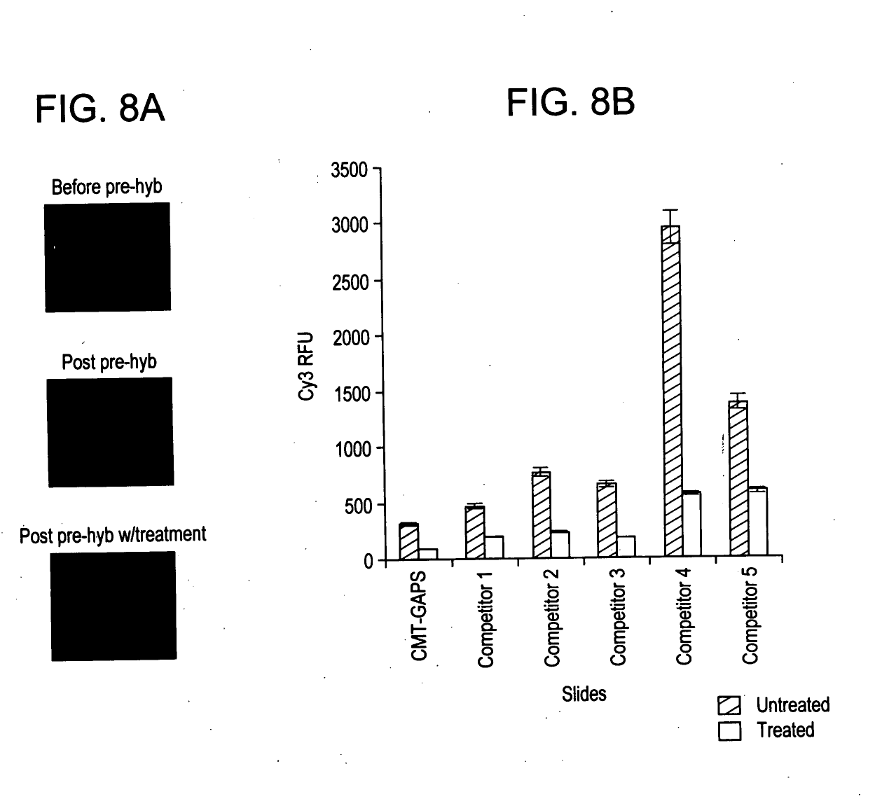

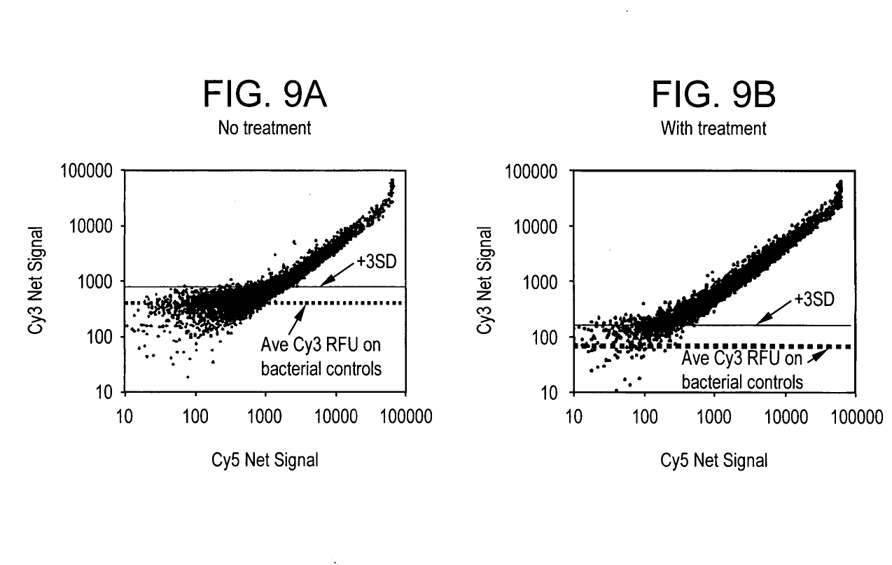

As described in U.S. patent application Ser. No. 09/925,808, and illustrated accompanying FIGS. 8 and 9, treatment of the microarray with the present background reducing solution before hybridization dramatically lowers the auto-fluorescence signal of the substrate surface and microspots. By reducing the background signal, as shown in FIG. 8, a superior array performance is highlighted in FIG. 9. FIG. 9 represents assay results performed on an array containing 5751 different human gene micropsots and 161 bacteria control spots representing nine different genes. Net signals are determined by subtracting local background from the raw signal intensity. The net signal on the bacteria control spots is mostly due to non specific hybridization. The average net signal of the control genes is shown as the dotted line, and the sum of net signal and three standard-deviations (3SD) is represented by the solid line. FIG. 9A shows an array that was not treated with the present background reducing solution. The number of spots in this array with relative fluorescence (RFU) less than the average signal of bacteria genes in Cy3-channel is 1008. In contrast, FIG. 9B show an array that was treated with the present background reducing solution. The number of spots with a relative fluorescence (RFU) less than the average signal of bacteria genes in Cy3-channel is only 263. This result indicates that the significant background reduction is achieved with treatment using the present invention, and the present invention provides a more reliable solution for end users to analyze genes that express on a low abundance or level.

### F. Pre-Hybridization Solution

Hybridization of target sequences to the nucleic acid microarrays occurs under conditions in which probe sequences on the microarrays are in excess relative to target sequences in a sample. In other words, gene expression profiling as well as SNP analysis, can be effective only when the number of each probe molecule available for target binding is much higher than the number of the target molecules in the sample. Therefore, any loss of the target sequences during the hybridization could significantly impact the success of the assays. Due to imperfections of probe immobilization on the substrate surface, a fraction of the probe molecules may be weakly attached or physical adsorbed, and could either come free of the microspots during the hybridization, or be washed away during the post-hybridization processes. These “free” probe molecules could bind to corresponding target sequences, and result in a further loss of accessible targets in the sample. Also, non-specific binding of the labeled target sequences to the surface of the substrate can result in a significant loss of available target sequences. A common approach used to eliminate these potential problems is to subject the microarray-bearing substrate to a pre-hybridization solution, which deactivates the surface of the slide surrounding each microspot. The pre-hybridization solution contains water-soluble blocker reagents that can form closely-packed layer(s) on the substrate surface to block non-specific binding of the targets. Moreover, treatment of the slides with a pre-hybridization solution beforehand can remove weakly attached or physical absorbed probe molecules.

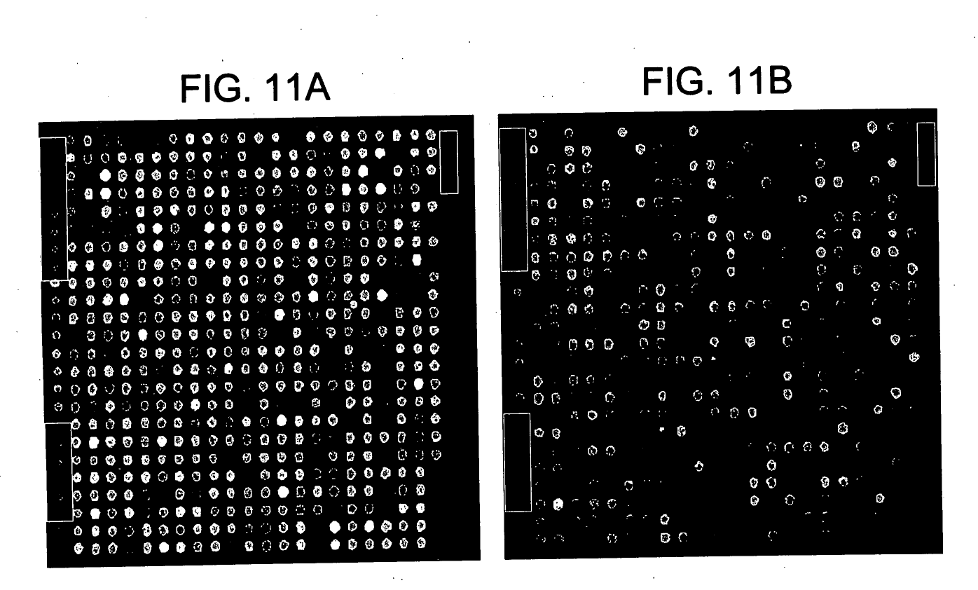

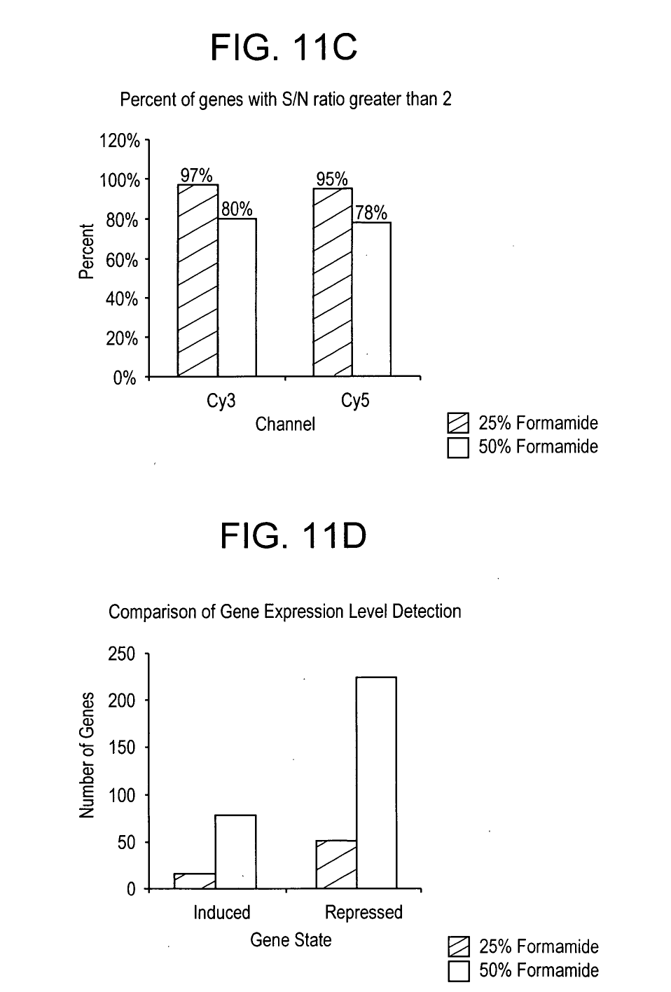

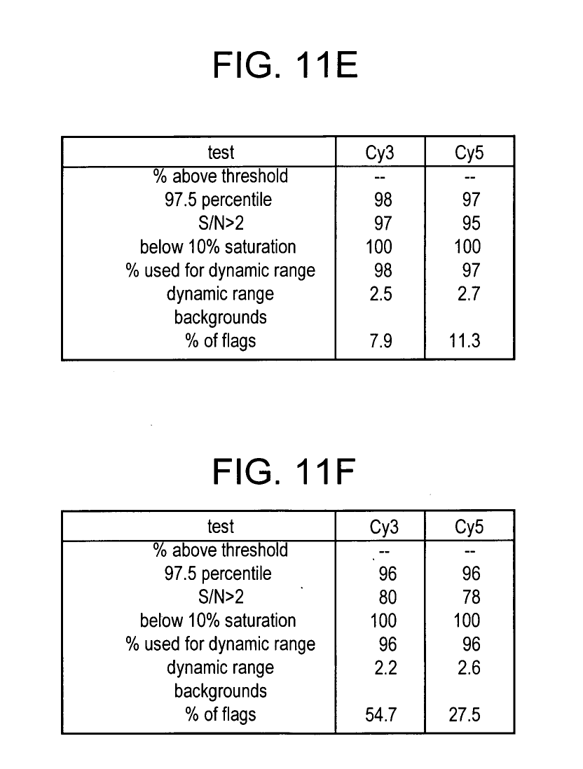

In terms of cDNA microarrays, the probe molecules are generally at least partially double-stranded. On the other hand, for oliognucleotide microarrays, the probe molecules might also adopt some types of secondary structures depending on the sequence and environmental conditions. Thus, DNA-denaturing reagents might be also included in the solution to transform probe molecules into single-stranded molecules in order to enhance the sequential hybridization efficiency. For example, as shown in FIG. 11, the presence of about 50% formamide in the pre-hybridization solution results in greater binding signals after assays compared to that achieved with about 25% formamide. In other words, when 50% formamide is used, the amount of non-specific binding that takes place is dramatically reduced. In contrast, when a 25% formamide formulation is used, everything fluoresces when it should not, for instance in FIGS. 11A versus 11B, as indicated by the microspots within the white boxes on the right and left periphery of the microarray. Higher specificity reduces the number spots with signal to noise ratio (S/N) of >2, as well as increases the level of detection in gene regulation (up or down).

The present pre-hybridization solution, according an embodiment for cDNA microarrays, comprises about: 20-70% vol. formamide, 0.1-5% wt. aqueous soluble protein (e.g., BSA) in buffer solution of 2×SSC at pH of about 6-9. Preferably the pre-hybridization solution composition includes about: 40-60% formamide, 0.2-1.7% aqueous soluble protein (e.g., BSA) in buffer solution of 2×SSC at pH of about 7-8.

Alternatively, the pre-hybridization solution, according an embodiment for either cDNA or oligonucleotide or both microarrays, comprises about: 0.1-5% wt. aqueous soluble protein (e.g., BSA), 0.05-5% vol. sodium lauryl sulfate (SDS) in buffer solution of 2×SSC at pH of about 6-9. Preferably the pre-hybridization solution composition includes about: 0.2-1.7% aqueous soluble protein (e.g., BSA) 0.1-1.5% vol. sodium lauryl sulfate (SDS) in buffer solution of 2×SSC at pH of about 7-8.

### G. Hybridization Solution

To develop hybridization assays in a microarray format, four parameters should be considered. These are: 1) specificity of interaction between a probe and its complementary target molecule, and the associated 2) stringency of the assay, as well as 3) hybridization efficiency, and 4) kinetics. Manipulation of these four parameters affects the quality of the assay results. Generally, a change in solvent, buffer formulation, or temperature will lead to modified stringency. Determination of the precise or particular formulation, however, is not a simple or easy task. A higher or tighter assay stringency in combination with the use of repeated sequences, for instance calf thymus DNA, Herring sperm DNA, Cot-1 DNA and/or poly-A, can give rise to greater specific binding between probe and target sequences. The presence of extra nucleic acids that contain repeating sequences, particularly Cot-1 DNA and/or poly-A, suppresses repetitive sequences in the target sample.

A buffer composition could cause the formation of at least partially double-helical or other secondary structures of the nucleic acid sequences themselves. By controlling the ionic strength and composition of the hybridization solution and the reaction time, one can achieve efficient hybridization between probes and targets. For instance, dextran sulfate is commonly used in Northern blotting or Southern blotting to increase the hybridization kinetics. To date, few DNA-microarray assay methods, however, have suggested the use of dextran sulfate during hybridization. The effect of dextran sulfate on array performance strongly depends on the reaction temperature, concentration of targets, other hydridization reagent components, and its own concentration. At low temperature and high DNA concentrations, or relatively high dextran sulfate concentrations (e.g., >10%), we found that both probe and target molecules tend to aggregate and precipitate, leading to uneven and high fluorescent background, and the assay stringency is reduced. As a result, overall hybridization specificity is decreased. On the other hand, we also found that hybridization with BSA can significantly reduce non-specific biding of probe on the GAPS-coated slide surface.

Accordingly, in an embodiment for the hybridization solution, the hybridization solution, according an embodiment for either cDNA or oligonucleotide or both microarrays, comprises about: 20-70% vol. formamide, 0.05-1.5% sodium lauryl sulfate (SDS), 0.1-5% wt. aqueous soluble protein (e.g., BSA), 1-10% dextran sulfate, 0.01-0.5 mg/ml poly-A, 0.1-50 μg/ml Cot-1 DNA, in buffer solution of 0.5-7×SSC at pH of about 6-9. Preferably the hybridization solution composition includes about 40-60% formamide, 0.05-0.5% sodium lauryl sulfate (SDS), 0.1-1.5% wt. aqueous soluble protein (e.g., BSA), 2-7% dextran sulfate, 0.05-0.25 mg/ml poly-A, 1-12 μg/ml Cot-1 DNA, in buffer solution of 0.5-2×SSC at pH of about 6.5-7.5.

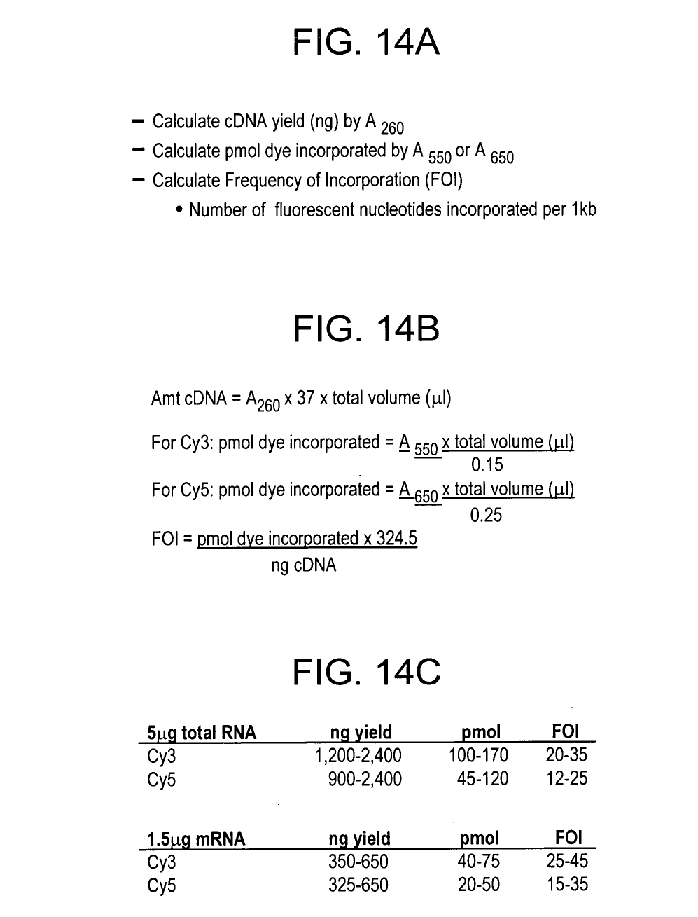

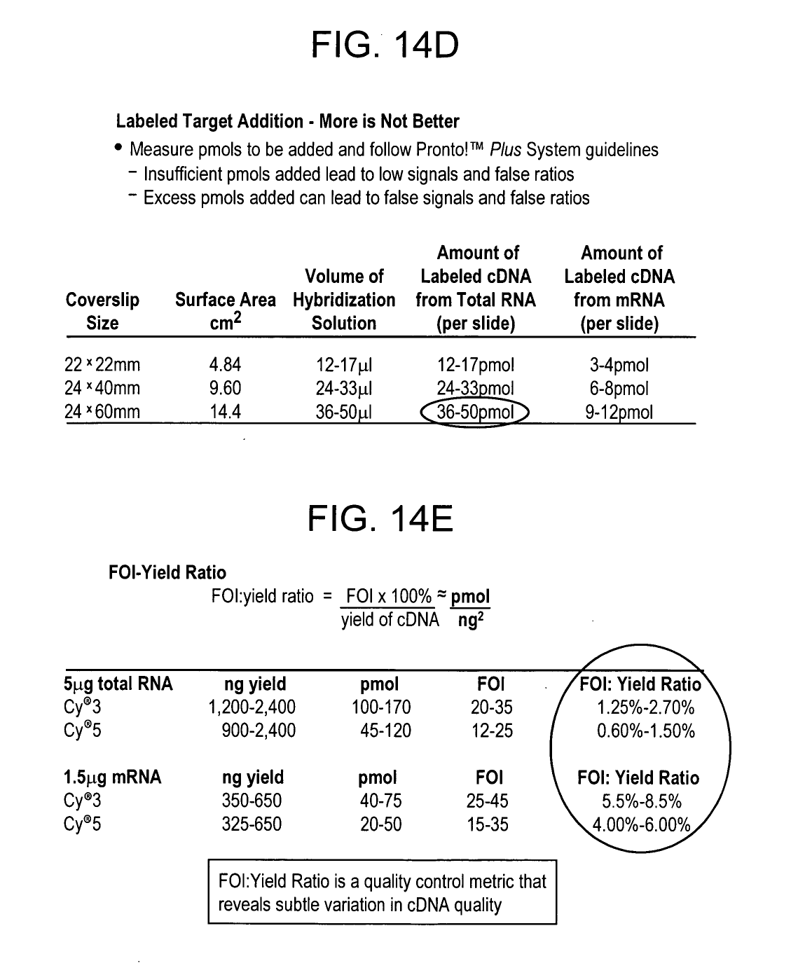

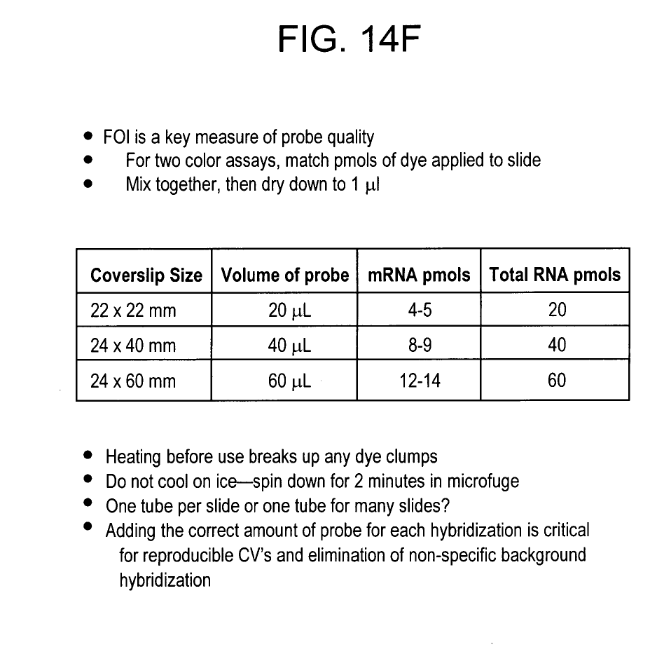

Addition of labeled cDNA to the hybridization solution should be performed in accordance with the guidelines as specified in the system configuration (FIGS. 14A-F) in order to achieve the greatest sensitivity, reproducibility, and consistency in terms of background and non-specific hybridization control. Amounts added for hybridization should be optimized based on pmol values as calculated using the formulations provided for yield, FOI, and FOI to Yield ratios. Volume for hybridization solution is dependent on the size of the coverslip used for hybridization.

### H. Wash Solutions

The wash step should be incorporated as an intermediate between each of the other steps in the assay protocol. Washing serves several purposes. First, the wash reduces interference associated with carrying over of solutions from a prior step into the subsequent step. Second, the wash removes unbound and/or weakly attached target molecules after hybridization, which presence would otherwise heighten background and obscure the sensitivity and specificity. Cross-reaction of the targets with non-complementary probes is relatively weak and can be suppressed by applying a stringent wash. The washing solutions have been formulated to reduce background signal and thus achieve the highest sensitivity.

In an embodiment, washing solutions according to the present invention comprises two separate solutions, Wash Buffer A and B, which are stored apart. Wash Buffer A contains about: 20×SSC (3M sodium chloride, 0.3 M sodium citrate-2H₂O, pH adjusted to 7.0 with hydrochloric acid); pH: 7.0±0.15. Wash Buffer B contains about: 10% of a surfactant (e.g., sodium dodecyl sulfate) in aqueous solution (preferably de-ionized water); pH: 5.5±0.15.

In a related embodiment, the washing solution used in a particular step of the protocol is reformulated. Generally, three washing solutions are prepared from Wash Buffer A and B. The three washing solutions are (1) Wash Solution 1:447.5 mls of deionized water (18 MegaOhm Milli-Q preferred), 50 mls of wash reagent A and 2.5 mls of wash reagent B; (2) Wash Solution 2:1425 mls of deionized water (18 MegaOhm Milli-Q preferred) and 75 mls of wash reagent A; (3) Wash Solution 3:300 mls of Wash Solution 2 and 1200 mls of deionized water (18 MegaOhm), such as from Milli-Q™. These three washing solutions may be used sequentially or combined as detailed in the examples below. It is extremely important not to allow the arrays to dry out between washes, as this will result in high backgrounds.

### I. Formulations and Storage of the Reagent Systems

The wide-spread use of DNA microarrray technology continuously drives the development and standardization of assays. The assay protocols have been streamlined and the end users are eager to adopt the standardized reagent systems in which are optimized according to the nature and uses of the microarrays. An ideal reagent system should also be easy and friendly to use and handle. Generally, the whole reagent system includes several reagent solutions, each used in a respective step of an assay protocol. Each reagent solution might include multiple critical components in order to achieve optimal performance. These components are preferably pre-mixed in a single container to the reagent solution and should be stable for certain periods of time. Due to the compatibility and stability of these components, however, separate storage of these components and pre-mixing by end-users right before the assays are industrial standard, and recommended by a number of commercial vendors.

The present invention overcomes these issues by optimizing the compositions in each reagent solution, wherein each reagent solution only contains necessary components, and each component is at carefully defined concentrations. The quality of the reagent components is an important factor, as well as selection of the appropriate filtering mechanisms used to purify the solutions of impurities or precipitates, which can later serve as ‘seeds’ for crystallization or precipitation of components. The pre-formulated reagent solutions according to the present invention can be stored under wide range of conditions, from cold or frozen conditions (e.g., temperature range from about −80° C. to about 5° C.) to ambient conditions and even higher temperatures. When the reagent solutions are stored at cold conditions, bringing the temperature of the solution to a temperature higher than ambient room temperature (˜20° C.), even briefly before use, can significantly enhance its performance and promote hybridization signal and specificity. For better assay results, one should preferably heat the reagent solutions before using to a temperature of about 38-48° C. for at least about 20 or 25 minutes to about 60 minutes.

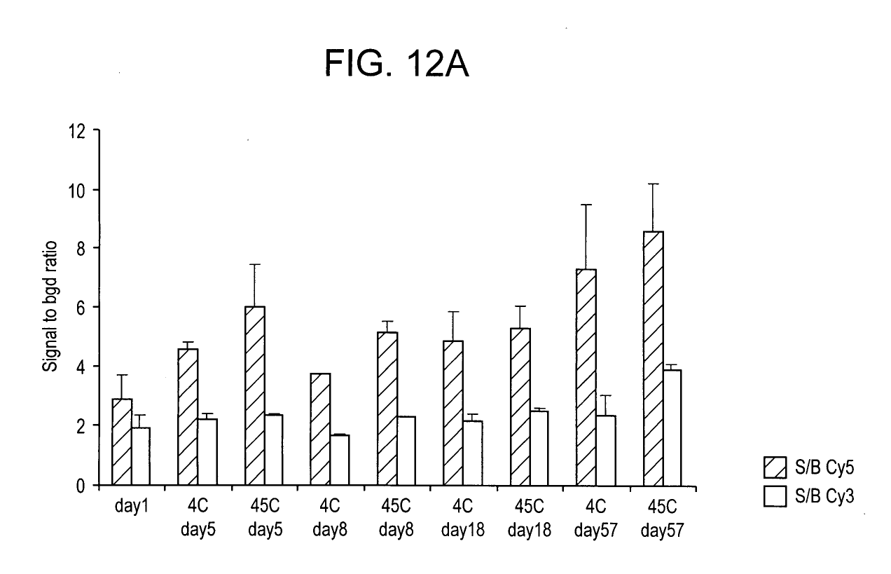

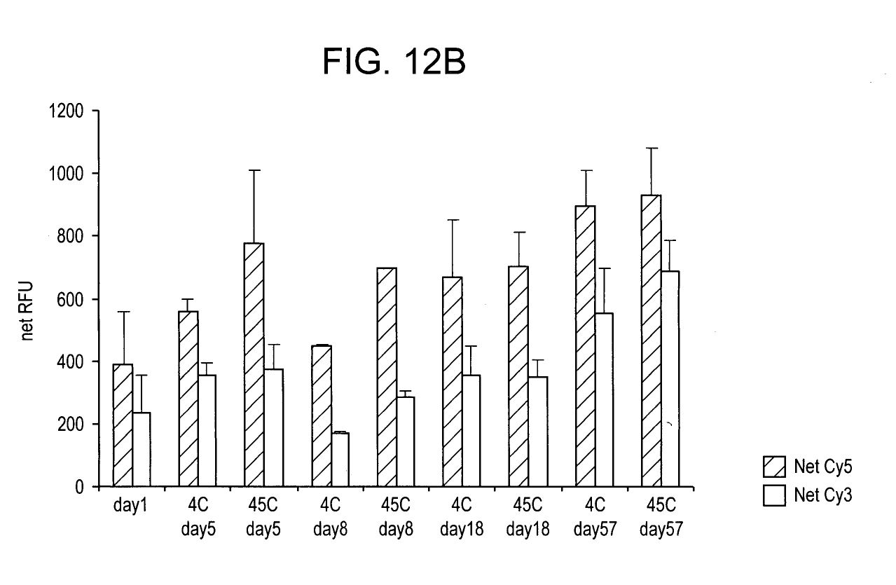

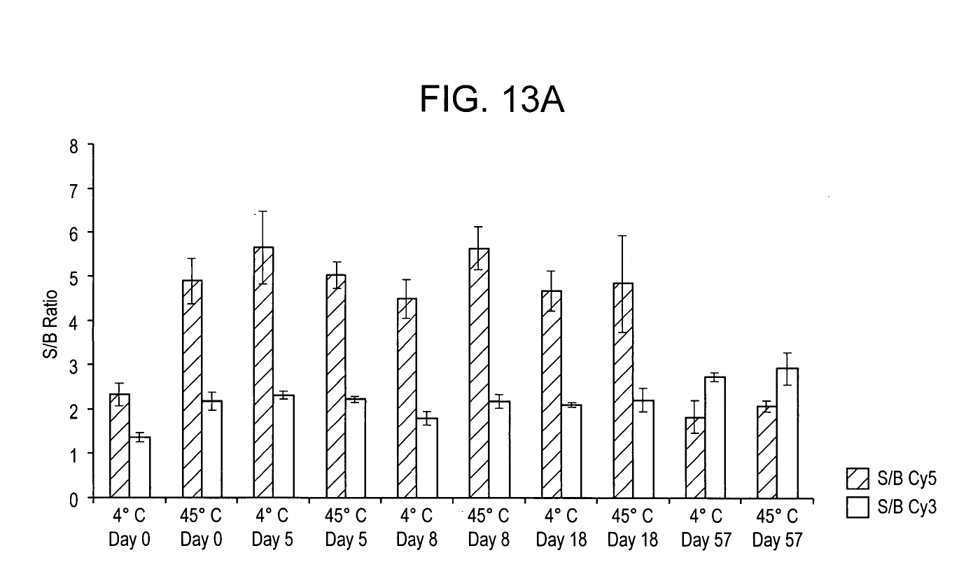

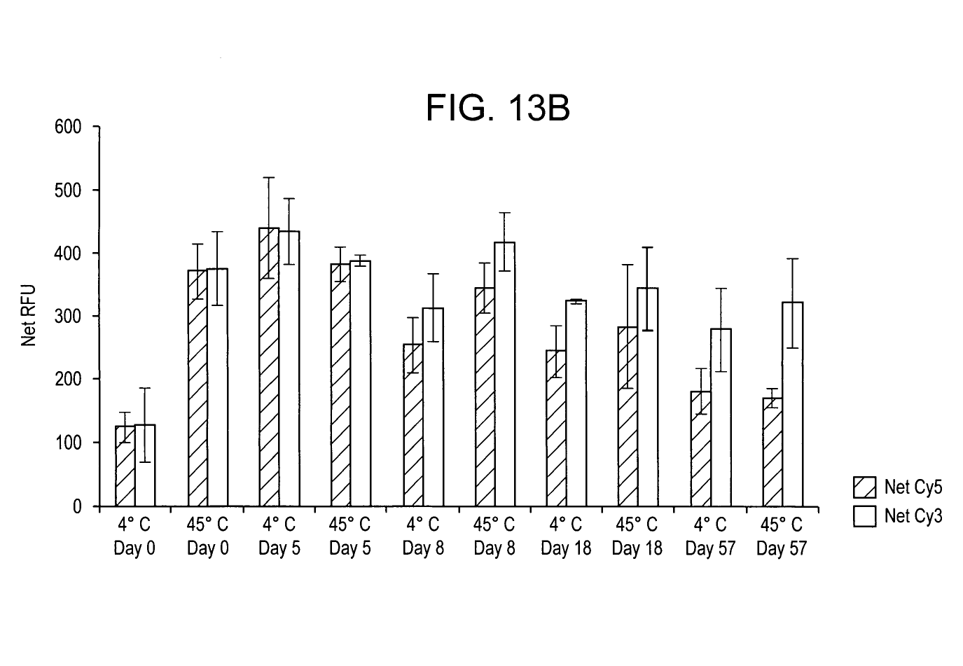

The various formulations of the present reagent solutions exhibit superior stability when stored over two months at temperatures greater than 4 or 5° C., as shown in FIG. 12 and 13. FIGS. 12A and 12B show the relative accelerated stability of hybridization studies carried out using a reagent kit according to an embodiment of the present invention. The y-axis in FIG. 12A presents data for the signal to background noise ratio, and in FIG. 12B presents data for net signal fluorescence. FIGS. 13A and 13B show the relative accelerated stability of hybridization studies using a reagent kit according to another embodiment of the present invention. The y-axis in FIG. 13A presents data for the signal to background noise ratio, and in FIG. 13B presents data for net signal fluorescence. These results confirm that the pre-formulated reagents of the reagent systems perform similarly under a long period of time under storage conditions.

According to an embodiment of the reagent kit, which promotes increased or high sensitivity, the pre-hybridization solution includes an optimized formulation of about 50% foramide, 5×SSC (0.75 M sodium chloride, 0.075 M sodium citrate 2H₂O, pH adjusted to about 7.0 with hydrochloric acid), ˜0.1% sodium dodecyl sulfate, ˜0.1 μg/μl low autofluorescence, bovine serum albumin (Fraction V cold ethanol precipitated, pH specification: 8.0±0.15, mV specification: 40 mS/cm±2.5.

In addition, the pre-formulated reagent solutions may each contain a particular formulation that not only enhances hybridization performance, but also preserves the biological component of each solution over periods of time for up to at least as long as a year. According to an embodiment of the invention, a hybridization solution may comprise, in terms of percent volume, about 35-95% water, up to about 5% or 7% of a low fluorescence protein-blocker molecule or complex of blocker molecules (e.g., bovine albumin), up to about 5% or 7% of a high molecular weight volume-excluder molcule and either about 35-65% formamide or ≦2% sodium lauryl sulfate. A hybridization solution may comprise about 35-65% water, 27-55% formamide, up to about 5% bovine albumin, and up to about 2% of a surfactant or detergent, anionic, cationic or non-ionic salt (e.g., sodium lauryl sulfate), and/or DNA oligomers, respectively. A post-hybridization wash may comprise a buffer solution having a pH in the range of about 6-8.

More comprehensive kits, according to another embodiment, in addition to the basic components already described, may contain enzymatic and/or biological reagents, which are to be stored separately from the kit at about −10° C. to −20° C. The enzymes or biological molecules may include: reverse transcriptase, Klenow-fragment or DNA-dependent polymerase, ribonuclease, or nucleic acid (e.g., primers; unlabeled or labeled nucleotides).

### SECTION III

### EXAMPLES

The following are illustrative examples of the reagent solutions, which further describe the present invention, its uses and advantages.

### Example I

### Fabrication of Oligonucleotide Arrays

1. Preparation of Oligonucleotide Probe Solution

Following one of alternative methods a) or b), below, DNA source plates (e.g., sterile, nuclease-free Corning 384-well Storage Plates; Cat. No. 3656) are prepared. Sufficient volume of printing solution needs to be prepared to cover the bottom of the receiving wells; this corresponds to between 5 and 10 μl per well when using 384-well plates.
a) Dissolve oligonucleotides to a maximum of 1.0 mg/ml (0.5 is a good starting concentrations for further optimization) in the spotting solution according to the present solution. Transfer DNA solution to Corning 384-well plate.
b) Alternatively, add the desired volume of the spotting solution to wells containing DNA that has been dried by vacuum centrifugation.

2. Array Fabrication Using Pin Printing Technology

To form a microarray in a desired configuration with desired density, according to manufacturer's or laboratory protocol, an arrayer device, available from various vendors (e.g., Cartesian Technologies, Gene Machines OmniGrid, BioRobotics, Seiko, Vertex, Genetix Microgrid, etc.), is set up to print the oligonucleotide probes onto a slide substrate. Preferably, the substrate is a high-quality glass slide, such as Corning UltraGAPS™), which has an ultra flat surface with an average roughness of less than about 10 nanometers (see e.g., U.S. Pat. No. 6,461,734) and a non-sodium borosilicate composition. The printing environment should be free of dust particles, and kept at a temperature of about 15-30° C., preferably about 20-27° C., and under relative humidity between about 30% and about 72%, preferably about 45% and about 55%. After printing, the microarray-bearing slides are placed in a desiccator for up to 48 hours (vacuum desiccator preferred), following by applying about 150-600 or 650 mJ of UV energy to the printed surface to cause the spotted oligonucleotides cross-linked to the substrate surfaces. The microarrays can be stored in a dry environment at normal laboratory temperature (20 to 25° C.). Arrays can be stored for at least 6 months prior to hybridization. Exchanging the regular atmospheric air for clean nitrogen gas helps prevent oxidation of spotted material and extends the shelf life of the arrays.

3. Results

FIG. 6 shows a graph of the evaporation rate of the spotting or printing ink according to the present invention, in comparison with six other commonly used spotting solutions. The spotting solution according to the present invention shows considerably lower evaporation rate. A lower evaporation rate allows researchers the flexibility to perform longer and more consistent printing runs for microarray fabrication.

### Example II

### Fabrication of Double-Stranded DNA Arrays

1. Preparation of Double Stranded Probe Solution

DNA source plates (sterile, nuclease-free Corning 384-well Storage Pates are recommended; Cat. No. 3656) are prepared by one of alternative methods a) or b), below. Sufficient volume of printing solution needs to be prepared to cover the bottom of the receiving wells; this corresponds to between 5 and 10 μl per well when using 384-well plates.
a) Dissolve dsDNA (typically Polymerase Chain Reaction amplified products generated from plasmid libraries that have been created using traditional cloning principles were RNA is the starting source material) to a maximum of 0.25 mg/ml (0.1 mg/ml is a good starting concentrations for further optimization) in the spotting solution according to the present solution. Transfer DNA solution to Corning 384-well plate.
b) Alternatively, add the desired volume of the spotting solution to wells containing DNA that has been dried by vacuum centrifugation.

2. Array Fabrication Using Pin Printing Technology

Similar to the process above, the arrayer device is set up to print the cDNA probes onto a high-quality glass slide, such as Corning UltraGAPS™. The printing environment should be free of dust particles, and kept at a temperature of about 13-32° C., preferably about 20-25° C., and under relative humidity between about 30% and 68%, preferably 45% and 55%. After printing, the microarray-bearing slides are placed in a desiccator for up to 48 hours (vacuum desiccator works best). Afterwards, the spotted DNAs in arrays are re-hydrated by holding slide (array side down) over a bath of hot dd-H₂O (95 to 100° C.) for approximately 5 seconds until condensation of the water vapor is observed across the slide, and snap-drying the arrays by placing it (DNA side up) on a hot plate for 2 sec. Then, the arrays are subjected to treatment eitherby 75 to 1200 mJ of UV light to the printed surface of the array, or by baking the array at 80° C. for 2 to 4 hours. The printed arrays can be stored in a dry environment at normal laboratory temperature (20 to 25° C.) for at least 6 months prior to hybridization. Exchanging the regular atmospheric air for clean nitrogen gas helps prevent oxidation of spotted material and extends the shelf life of the arrays.

3. Results

The use of the spotting solutions results in enhanced printing quality and hybridization performance. As shown in FIG. 2, the spotting solution according to the present invention is used to fabricate arrays of cDNAs on Corning UltraGAPS™ slides. FIG. 2 compares a series of fluorescence images for nucleic acid microarrays on different substrates. Column A represents an array prepared on Corning UltraGAPS™ slides and Columns B-D, each represents an array printed on three other commercially available amine-presenting substrates. An assay is performed on each microarray. Column A presents images obtained at three major points of the assay protocol according to the present invention. Columns B-D represents corresponding images for assays according to the respective vendor's recommended protocol. The resulting arrays in Column A show uniform spot size and consistent morphology throughout the assays, and low auto-fluorescence background before and after hybridization. The binding of the targets to their corresponding probe microspots shows high specificity and reproducibility with high assay sensitivity (high signal-to-noise ratio). In contrast, following corresponding protocols from different commercial vendors, using three other spotting solutions, the arrays and assays in Columns B-D resulted in a high failure rate of array fabrication, due to either high auto-fluorescence signals from the microspots and low assay sensitivity (B), low binding signals of targets to the probe microarrays (C), or relatively high auto-fluorescence signals from the microspots and undesired spot morphology (D).

### Example III

### Target Nucleic Acid Sequence Labeling

(Note: All Cy labels below are a trademark of Amersham, i.e. Cy™)

1. Genomic DNA Labeling Using a Solution Containing Random Primers

To label genomic DNA, a 28-29 μl solution containing 4 μg of human gDNA and 3.6-4 μg of random hexamers in a buffer solution according to the present invention is incubated at 95° C. for 5 minutes, briefly chilled on ice, and then added to a 11-12 μl solution containing approximately: 4 μl of 10× EcoPol buffer; 2 μl of 0.1 M DTT; and 1.5-3 μL of a dNTP mixture. The dNTP mixture consists of 10 mM each of dGTP, dATP, dTTP, and 1 mM of dCTP; 2 μl of Cy3- or Cy5-dCTP at 1 mM (PerkinElmer, Boston, Mass.); and 1-1.2 μl of Klenow fragment (New England Biolabs, Inc., Beverly, Mass.). The combined total 40 μl solution is incubated at 37° C. for about two hours. The labeled target is purified using a standard PCR purification methods (e.g., Qiagen, Inc., Valencia, Calif.). The cDNA concentration and the amount of Cy3/Cy5 incorporation is measured on an Agilent 8453E UV-Vis spectrometer.

2. mRNA Labeling Using a Solution Containing Random Primers

Messenger RNA labeling generally involves incorporating a Cy-dye with cDNA synthesized by reverse transcription of mRNA in the presence of Cy-dCTP. Using random primers following the present invention, Cy-dCTP incorporation rates can be achieved in good yields and high consistency with cDNA labeling systems from several suppliers (e.g., SuperScript II system (Invitrogen) and FluoroLink Cy3-and Cy5-dCTP (AP Biotech) for Cy-cDNA synthesis, and the QIAquick PCR columns (Qiagen)) and standard DNA purification methods for Cy-cDNA purification of the Cy-cDNA.

To label mRNA in a sample, a ˜23 μl solution containing 1.5 μg of mRNA sample and 3.6-4 μg of random hexamers in a nuclease-free water according to the present invention is incubated at 70° C. for 10 minutes, briefly chilled on ice, and then added to a ˜17 μL solution containing approximately: 4 μl of 10× Superscript II buffer (Invitrogen); 4 μl of 0.1 M DTT; and 1.5-3 μl of a dNTP mixture; ˜4 μl Superscript II (Invitrogen, 200U /μl). The dNTP mixture consists of 10 mM each of dGTP, dATP, dTTP, and 1 mM of dCTP; 2 μl of Cy3- or Cy5-dCTP at 1 mM (PerkinElmer, Boston, Mass.); and 1-1.2 μl of Klenow fragment (New England Biolabs, Inc., Beverly, Mass.). The combined total 40 μl solution is incubated at 42° C. for about two hours. The labeled target is purified using a QIAquick PCR purification kit according to the manufacturer's instructions (Qiagen, Inc., Valencia, Calif.). The cDNA concentration and the amount of Cy3/Cy5 incorporation is measured on an Agilent 8453E UV-Vis spectrometer.

3. Total RNA Labeling Using a Solution Containing Both Oligo dT Primers and Random Primers

To label total RNA in a sample, a ˜23 μl solution containing 5 μg of mRNA 5, 3.0-4 μg random hexamers, optionally with ˜2 μg oligo dT primer in a nuclease-free water according to the present invention is incubated at 70° C. for 10 minutes, briefly chilled on ice, and then added to a ˜17 μl solution containing approximately: 4 μl of 10× Superscript II buffer (Invitrogen); 4 μl of 0.1 M DTT; and 3˜μl of a dNTP mixture; ˜4 μl Superscript II (Invitrogen, 200 U /μl). The dNTP mixture consists of 10 mM each of dGTP, dATP, dTTP, and 1 mM of dCTP; 2 μl of Cy3- or Cy5-dCTP at 1 mM (PerkinElmer, Boston, Mass.); and 1-1.2 μl of Klenow fragment (New England Biolabs, Inc., Beverly, Mass.). The combined total 40 μl solution isincubated at ambient temperature for 10 min, followed by incubation at 42° C. for about two hours. The labeled target is purified using a QIAquick PCR purification kit according to the manufacturer's instructions (Qiagen, Inc., Valencia, Calif.). The cDNA concentration and the amount of Cy3/Cy5 incorporation is measured on an Agilent 8453E UV-Vis spectrometer.

4. Purification of Labeled Target Sequences

After RNAse treatment by adding 1.0 μl RNAse H (InVitrogen, 1-4 U/μl ) and 0.25 μl RNAse A (USB, 20-30 U/μl into the cDNA-synthesis reaction and sequential incubation at 37° for 15 minutes, ethanol precipitation with sequential purification using QIAquick PCR purification columns and reagents is used to purify labeled target sequences.

5. Results

In FIG. 7, a set of DNA targets for a B. subtilis gene is deposited on an array. A sample of a 1.2 kb B. subtilis RNA (with an engineered poly-A tail) was produced using in vitro transcription. For the set of tiling oligonucleotides, 4 oligonucleotides (60mers) are synthesized to cover the whole length of the RNA molecule. Each oligonucleotide was 300-400 nt apart. These oligonucleotides are printed on GAPS slides as probes. RNA is labeled by reverse transcription with either poly-dT primer or semi-random primers. The hybridization results show that both the Cy3 and Cy5 signal with poly-dT labeled probe is similar to random primer labeled probe near the 3′ end of RNA, indicating both primers work with similar efficiency. The hybridization signal, however, dropped significantly for the targets near the 5′-end with poly-dT primer labeled probe, as depicted in FIG. 7. This reflects the reduced transcription efficiency of the 5′ end compared to the 3′ end and reveals an advantage of using random primer over poly-dT primers during reverse transcription.

### Example IV

### Treatment with Pre-Soak Solution to Reduce Background

1. Treatment of the Prepared Array

Following the optimal assay protocols according to the present invention, the effect of the treatment of the microarrays using the background reducing solution before hybridization is evaluated using cDNA microarrays in combination with lung RNA samples. The printed array-bearing slides are subject to the treatment using the pre-soak or background-reducing solution according to the present invention. The treatment is carried out in a 100 ml staining jar that can potentially hold up to five slides. The staining jar is filled up with 100 ml of pre-heated pre-soak solution (˜42° C.), followed by completely dissolving one 0.25 g tablet of NaBH₄. Once the NaBH₄ tablet is completely dissolved, array-bearing slides are removed from package, and added to the pre-soak Solution one at a time. Afterwards, the slides are pre-soaked at 42° C. for 20 minutes. Once removed from staining jar, the slides are placed into a staining jar filled with 100 ml of the washing solution and incubate at room temperature for 30 seconds. After repeating the above washing step twice more in two new 100 ml staining jars using the wash solutions, the slides are transferred, and subject to the sequential incubation with the pre-hybridization solution, the washing solution, the hybridization solution, the wash solution again and finally dried and examined with a scanner.

2. Results

FIG. 8 is a demonstration, according to the present invention, of the effective reduction of auto-fluorescence background of the microarray and its substrate surface using a reducing reagent such as borohydride. The three images A-C, highlight the dramatic reduction in background after treatment with the reducing reagent. The graph D summarizes the statistical results of the present solution in comparison with microarrays on five different substrate surfaces from five commercial vendors Results show that for microarrays on all surfaces tested, the auto-fluorescence background of the surface and the microspots in both Cy3 channel and Cy5 channel (data not shown) could be significantly compressed.

FIGS. 9A and 9B are graphs showing the net signals of both Cy3 and Cy5, respectively, of a human 6k microarray after self-self hybridization with human brain RNA, with or without being treated with a background reducing solution according to the present invention. The array contains 5751 different human target spots and 161 bacterial control gents. The average of non-specific binding signals of the bacterial control genes is used to define the noise level (i.e., total background baseline). As one can see in the graphs, the total background baselines in the both channels are much lowered for microarrays treated with the background reducing solution. Furthermore, as the results of the background reduction, the sensitivity to expression data is significantly enhanced, signal correlation between the two fluorophores is improved, and the dynamic range of signal intensity is broadened, as evidenced by the greater number of genes having signals above the average signals of control bacterial genes.

### Example V

### Hybridization Solution Enhances Array Performance

1. Assays

Human 4k cancer microarrays are used to examine the effect of dextran sulfate in the hybridization solution on gene profiling. Following the pre-soaking and pre-hybridization, the microarrays are subject to hybridization with a specific amount (aliquoit) of labeled cDNA generated from a starting amount of ˜5.0 μg lung total RNA in a defined volume of the hybridization solution in which is in the absence and presence of high molecular-weight dextran sulfate. The volume of hybridization solution needed depends on the size of the printed area and cover glass. One may use 2.5 μl of hybridization solution per cm² of surface area for a regular cover glass.

For the lung total RNA, Cy-cDNAs made from total RNA use 1.0 pmoles of incorporated nucleotides per microliter of hybridization solution, per dye. For example, to hybridize an area covered by one Corning 22×22 mm cover glass (approximately 5 cm²), dissolve an amount of total-RNA-derived cDNA containing 12 pmoles of each Cy3- and Cy5-dCTP in 12 μl of the hybridization solution.

For hybridization, the following protocol may be used:
(1) Wash the required number of pieces of cover glass (at least 1 piece of cover glass per array should be processed) with nuclease-free water, followed by ethanol. Dry cover glass by nitrogen flow or allow to air-dry in a dust-free environment.
(2) Dissolve the appropriate amount of fluorescently labeled cDNA in the required volume of the hybridization solution with or without Dextran sulfate at a concentration of 6% (weight/volume).
(3) Incubate the cDNA solution at about 95° C. for 5 min.
(4) Briefly centrifuge the cDNA to collect condensation, and allow it cool to room temperature. Do not place the solution on ice, as this will cause precipitation of some of the components.
(5) Deposit probes onto the surface of the printed side of the slide. Carefully place the cover glass on the array. Avoid trapping air bubbles between the array and the cover glass. Small air bubbles that do form usually dissipate during hybridization.
(6) Place the array in a hybridization chamber (e.g., Corning Cat. No. 2551).
(7) Incubate a chamber-array assembly at ˜42° C. for 12 to 16 hrs, using a water bath or a hybridization oven.

After hybridization, the microarray-bearing slides are subject to stringent wash using the wash reagents according to the present invention. Three wash solutions should be prepared before assays: (1) Wash Solution 1:447.5 mls of deionized water (e.g., 17-18.2 MegaOhm) (e.g., Milli-Q UltraPure™), 50 mls of wash reagent A and 2.5 mls of wash reagent B. (2) Wash Solution 2:1425 mls of deionized water (18 MegaOhm) and 75 mls of wash reagent A. (3) Wash Solution 3:300 mls of Wash Solution #2 and 1200 mls of deionized water (18 MegaOhm). It is extremely important not to allow the arrays to dry out between washes, as this will result in high backgrounds.

For post-hybridization wash, multiple containers are needed to perform the washes in the most efficient manner. Have all containers and the volumes of washing solutions ready before starting the procedure. The follow protocol is generally used:
(1) Fill one ajar with 100 ml of Wash Solution 1, pre-warmed to about 42° C. (this wash could be used to treat up to five slides at once);
(2) Open hybridization chamber, carefully remove and place arrays in staining jar;
(3) Remove the cover slips and allow slides to incubate at about 42° C. for 5 minutes;
(4) Transfer the slides into a second staining jar with 100 ml of Wash Solution 1 and incubate at 42° C. for 5 minutes;
(5) Transfer slides to a third staining jar with 100 ml ofWash Solution 2 and incubate at room temperature (˜20° C.) for 10 minutes;
(6) Transfer slides to a forth staining jar with 100 ml of Wash Solution3 and incubate at room temperature for 2 minutes;
(7) Repeat above wash step with Wash Solution 3, twice more in two new staining jars;
(8) Dry immediately under heavy stream of high purity nitrogen gas with the backside first, (the quicker the slide dries, the less chance of water spots on the array). Alternatively, dry slides by spinning at a low speed (e.g., 2000-2500 rpm), for 1 minute at room temperature.
(9) Store slides in a light proof container until ready to scan.
(10) Scan at appropriate settings.

2. Results

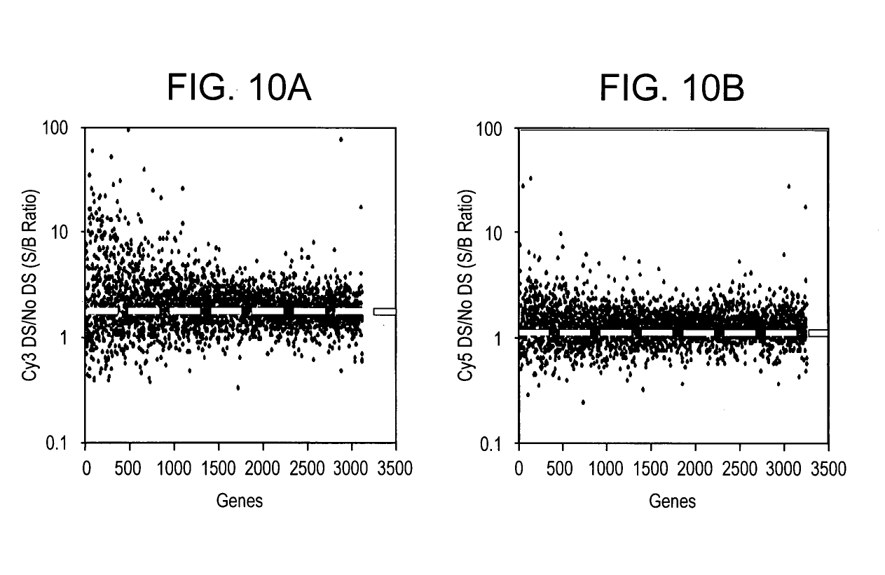

FIGS. 10A and 10B are graphs showing the ratio between the signal-to-background ratio of 3K genes on a microarray after self-self hybridization with a speficic amount of labeled cDNA generated from a starting amount of ˜4.54 μg of total testis RNA, in the presence of dextran sulfate (6%) and that obtained in the absence of dextran sulfate in the hybridization buffer solution. The addition of dextran sulfate (DS) should improve signal-to background ratios because it not only increase the viscosity of solution but also increases the local concentration of target nucleic acids near the probes on the surface. Results clearly confirm this expectation: in both Cy3 and Cy5 channels, the presence of the dextran sulfate in the hybridization solution improves the signal-to-background (S/N) ratio by 2.6 fold and 1.4 fold in average, respectively.

### Example VI

### Pre-Hybridization Solution Containing 50% Formamide Improves Dynamic Range for cDNA Microarrays

1. Assays

Human 2k cancer microarrays are used to examine the effect of formamide in the pre-hybridization solution on gene expression profiling. The pre-hybridization solutions used are similar, except that the concentration of formamide is different from 25% to 50% (volume/volume). Following the pre-hybridization, the microarrays are subject to the same hybridization and post-hybridization processes. The target sample is generated from ˜5.0 μg of lung total RNA, and is labeled with Cy3 and Cy5 (self-self hybridization).

According to an embodiment, the protocol of the pre-hybridization using the pre-hybridization solution is as follows:
(1). Prehybridization is performed in a 50 ml Coplin jar that holds a number of slide (e.g., 5 slides).
(2). Preheat 50 ml of cDNA Prehybe Solution to 42° C. in Coplin jar prior to adding slides. This should be done at least 30 minutes ahead of time.
(3). Carefully remove slides from package and add slides to cDNA Prehybe Solution one at a time.
(4). Up to five slides may be placed in Coplin jar, however be sure the slides on either edge are facing away from the side of the jar.
(6). Prehybe at 42° C. for 1 hour.
(7). Remove slides from Coplin jar one at a time.
(8). Rinse both sides of slide with gently running water for 5 seconds, to remove SDS from slide.
(9). Swish in 100% Ethanol for 2 seconds.
(10). Dry immediately under heavy stream of high purity nitrogen gas to remove ethanol, back of slide first. The faster you dry the slide, the less chance of water spots on the array. Alternatively, dry slides by spinning at low speed 2000-2500 rpm, for 1 minute at room temperature.

2. Results

FIGS. 11A and 11B are two scatter plots between integrated Cy3 signal versus Cy5 signals for genes in the microarrays after self-self hybridization with lung total RNA samples. The results show the slight improvement in correlation number (R²) between binding signals between Cy5 and Cy3 channels for microarrays treated with a pre-hybridization solution containing 50% formamide, compared to that with a solution containing 25% formamide. This result confirms the ability of formamide to denature double-stranded DNA, as well as the presence of at least partial double-stranded DNA in the probe microspots that can reduce the efficiency of the probe molecules hybridizing with their complementary target sequences.

### Example VII

### Long-Term Storage of the Reagent System

1. Results

Two different formulations for the reagent system are examined. Human 2K cancer arrays in combination with the lung mRNA samples are used as model systems to evaluate the reagent systems. FIG. 12 shows the results of accelerated stability studies using a so-called universal kit. These studies were carried out in polypropylene bottles at 4° C. and 45° C. and evaluated at day 1, day 5, day 8, day 18 and day 57 respectively. No significant difference in signal-to-background ratio (FIG. 12A) or net signal (FIG. 12B) was observed between the days and temperatures tested. At all four points of time, the signal-to-background ratio obtained using kits stored at 45° C. performed consistently equivalent to or better than counter parts stored at about 4° C. The data showed extremely good stability and assay performance. The projected shelf life of the universal kit from these studies is over 1 year at room temperature.

FIG. 13 shows the results of the same accelerated stability studies using a so-called cDNA kit. No significant difference in signal-to-background ratio for Cy3 channel for all time points tested while for Cy5 channel up to 18 days (FIG. 13A). Day-to-day experiment error may cause the slight variation in net signal (FIG. 13B). The cDNA kit may be stored at about 45° C. consistently performed equivalent or better than counter parts that were stored at about 4° C.

### Example VIII

### Improved Assay Performance for DNA

1. The reagent system

The reagent system used in this example includes a probe spotting solution, a target labeling solution, a pre-soaking solution, a pre-hybridization solution, a hybridization, a wash reagent A, and wash reagent B. Table 2 lists some examples of reagent solution compositions for the present reagent system.

TABLE 2
Reagent solutions compositions.
Spotting Solution

| Component | Percent |
|---|---|
| Ethylene glycol (EG) | ˜60-100 |

cDNA Spotting Solution

| Component | Percent |
|---|---|
| Water | ˜45-55 |
| Dimethyl sulfoxide (DMSO) | ˜45-55 |

Pre-Hybridization Solution

| Component | Percent by volume |
|---|---|
| Water | ˜85-95 |
| Albumin, bovine, fraction V | 4 |
| Sodium lauryl sulfate | <1 |

Hybridization Solution

| Component | Percent by Volume |
|---|---|
| Water | ˜50-60 |
| Formamide | ˜30-40 |
| Albumin, bovine, fraction V | <5 |

cDNA Pre-Hybridization Solution

| Component | Percent by Volume |
|---|---|
| Water | ˜40-60 |
| Formamide | ˜40-60 |
| Albumin, bovine, fraction V | <1 |

cDNA Hybridization Solution

| Component | Percent by Volume |
|---|---|
| Formamide | ˜45-50 |
| Water | ˜40-50 |
| DNA Oligomer(s) | <1 |
| Sodium lauryl sulfate | <1 |
| Albumin, bovine, fraction V | <1 |

Sodium Borohydride Pre-Soak Tablets

| Component | Percent |
|---|---|
| Sodium Borohydride | 100 |

Pre-Soak Solution

| Component | Percent |
|---|---|
| 2 × SSC | >99 |
| sodium lauryl sulfate | <1 |

Wash Reagent A

| Component | Percent |
|---|---|
| 20 × SSC | 100 |

Wash Reagent B

| Component | Percent |
|---|---|
| Water | 85-95 |
| sodium lauryl sulfate | 5-15 |

These reagent solutions can be pre-mixed and stored at room temperature. For optimal results with DNA microarrays assays, these reagent solutions may require additional treatment (e.g., pre-heat the solution before use), or be employed to modify or reformulate other reaction materials including probe sequences and target sequences in order to achieve optimal performance of the assays.

2. Assay Protocol

The following assay protocol is related to the method for performing RNA expression analysis using DNA microarrays. After each step, the microarrays can be imaged to examine the array quality as well as assay results. The RNA samples are obtained from commercial vendors, such as Qiagen, or Invitrogen. A self-self hybridization refers to a hybridization assay using equal amount of both Cy3- and Cy5-labeled target sequences driven from same RNA sample. For differential gene expression analysis, a hybridization involves an assay using equal total amount of two RNA samples, one from a abnormal tissue (such as cancers) labeled in one cobr, and the second one from a corresponding normal tissue labeled in a second different color.

(1) Target Labeling and Purification

For RNA target labeling, cDNA targets are synthesized from the total RNA by reverse transcription using a solution system based on the target labeling solution according to the present invention. The target labeling solution contains random oligo-primers in the absence and present of oligo-dT primers for mRNA and total RNA labeling, respectively. The labeling nucleotides are incorporated into the sample cDNA targets during the reverse transcription using the either a green fluorescent dye-tagged dCTP (e.g., Cy3-dCTP) or a red fluorescent dye tagged dCTP (e.g., Cy5-dCTP) according to the assay requirement. The labeled cDNA targets are mixed with reference sample before the assays. Certain amount of Cot-1 DNA is also included to suppress repeat sequences. During this step, several additional reagents are included in the labeling reaction except of the target labeling solution. They are human total RNA (Clonetech); Superscript II reverse transcriptase, DTT, RNAse H, RNase A, human Cot 1 DNA (Life Technologies); Cy3-dCTP, Cy5-dCTP (NEN), RNAse A (USB), QIAquick PCR purification kit (Qiagen) and poly A (Sigma).

To label total RNA, about 5 μg of total human RNA is used during primer-annealing. To two 1.5 ml micro-centrifuge tubes, one for Cy3 labeling and one for Cy5 labeling, the following components are added.

| | Cy3 | Cy5 |
|---|---|---|
| Total human RNA (5.0 μg/μL) | 1 | 1 μl |
| The target labeling solution | 21.5 | 21.5 μl |
| Total volume | 22.5 | 22.5 μl |

The RNA sample is then incubated at about 70° C. for 5 minutes, followed by a quick chill on ice. Subsequently, a reverse transcription labeling mixture consisting of the following is added to each tube.

| | Cy3 | Cy5 |
|---|---|---|
| 1) 5X Superscript II buffer (BRL) | 8 | 8 μl |
| 2) DTT (100 mM) | 4 | 4 μl |
| 3) dNTP mixture | 2 | 2 μl |
| 4) Cy3-dCTP (1 mM) | 1.5 | 0 μl |
| 5) Cy5-dCTP (1 mM) | 0 | 1.5 μl |
| 6) RT Enzyme (BRL) | 2 | 2 μl |
| Total volume | 17.5 | 17.5 μl |

The dNTP mixture consists essentially of a mix of 10 μl of 100 mM dGTP, 10 μl of 100 mM dATP, 10 μl of 100 mM of dTTP and 10 μl of 10 mM of dCTP, 60 μl of RNase/DNase free water, total volume 100 μl. The reverse transcription labeling mixture is added to the tube with annealed RNA, and mixed by vortex for about 10 seconds and spun for 10 seconds. Reverse transcriptase is then added and mixed well. The RNA is then incubated first at room temperature for 10 minutes, then at 42° C. for 2 hours. About 1 μl of RNase H and about 0.25 ul of RNase A, are added to degrade the RNA and incubated at 37° C. for 15 minutes. Subsequently, the probe material is purified using Qiagen's PCR purification kit. Five volumes of buffer PB (˜200 μL) is added to one volume of the labeling reaction (˜40 μl) and mixed. A QIAquick spin column is then placed in a 2 ml collection tube. To bind DNA, the sampleis applied to the QIAquick column and centrifuge for 60 seconds (14000 rcf) at RT (25° C.). To wash, about 600 μl of Buffer PE is added to the QIA quick column and centrifuged for 60 seconds (14000 rcf) at room temperature (˜20-25° C.). The wash is repeated for another 3 times. The flow-through is discarded and the QIAquick column is placed back in the same tube. The column is then centrifuged for an additional 60 seconds (14000 rcf) at room temperature (˜20-25° C.). To elute the cDNA probes, about 30 μl of 0.5× Buffer EB (5 mM Tris-Cl, pH 8.5) is added to the center of the QIAquick membrane, and the column is let to stand for about 1 minute. Then, the column is centrifuged for about 60 seconds (14000 rcf) at room temperature (25° C.). An elute volume of about 28 μl and the cDNA concentration and fluorescent dye incorporation (net 260, 280, 550, 650 OD with 480 OD for background subtraction) is measured. Using a Speed Vac, the volume of probe is reduced from 28 μl to about 5-8 μl.

(2) Array Fabrication Using the Reformulated Probe Sequences

For printing, the probe sequences are reformulated into an appropriate concentration (generally in the range of 0.01-2 mg/ml, preferably 0.5 mg/ml for oligonucleotides, 0.1 mg/ml for cDNA) using the probe spotting solution according to the present invention. The reformulated probes are ready for array fabrication.

(3) Background Reduction Using the Pre-Soak Solution

For background reduction, a microarray slide substrate is treated with the pre-soaking solution after a reducing agent, NaBH₄, is added. The treatment is carried out in a 100 ml staining jar that can potentially hold up to 5 slides. The staining jar is filled up with 100 ml of pre-heated pre-soak solution (˜42° C.), followed by completely dissolving one 0.25 g tablet of NaBH₄. Once NaBH₄ tablet is completely dissolved, array-bearing slides are removed from package, and added to the pre-soak Solution one at a time. Afterwards, the slides are pre-soak at 42° C. for 20 minutes. Once moved from staining jar one at a time, the slides are placed into a staining jar filled with 100 ml of the washing solution and incubate at room temperature for 30 seconds. After repeat above wash step with the wash solution twice more in two new 100 ml staining jars, the slides are transferred, and subject to the sequential assay steps.

(4) Pre-Blocking the Microarrays Using the Pre-Hybridization Solution

Following the pre-soaking step, the slide is transferred to another Coplin jar filled with about 100 ml of the pre-hybridization solution according to the present invention and incubated at 42° C. for 15 minutes. The pre-hybridization solution, 2×SSC/0.05% SDS/0.2% BSA, is pre-warmed up to 42° C. in a water bath (it takes about 20-30 min) The slide was transferred to a Coplin jar filled with 1× wash reagent A at room temperature for 1 minute, and again to a Coplin jar filled with 0.2× wash reagent A at room temperature for 1 minute. This step was repeated twice more. The slide was then spin-dried at 2000 rpm for 1 minute at 25° C.

(5) Hybridization Using the Hybridization Solution Containing the Target Sequences

Two possible approaches are considered in preparing the hybridization solution for human cDNA or oligonucleotide array platforms. In one approach, the target sequences are dissolved or resuspended in the present hybridization solution to an appropriate concentration. For instance, both Cy3-target and Cy5-target (each 5 μg of total RNA input) are combined in a 1.5 ml microcentrifuge tube containing about 60 μl the present hybridization solution. The targets are then denatured at 95° C. for 3 minutes, spun at room temperature for 30 seconds, and incubated at 42° C. for 2 minute. Afterwards, the solution is applied to the slide, and the targets in the solution are hybridized to the probes on the microarray. A 24 mm×60 mm cover-slip is then placed onto the array, mindful to avoid bubbles. The slide is then placed in a hybridization chamber comprised of a sealed pipette-tip box with 5×SSC buffer on the bottom. The microarray is placed immediately into an incubator at 42° C. for overnight of about 14-20 hours.

After incubation, the microarray is washed, without permitting the microarray to dry between individual washes. The microarray is immerse immediately in the present Washing Solution 1 contained in a 1^{st} Coplin jar (jar #1), at 42° C. for 1 minutes. In a series of washes, the slide is then transferred into a 2^{nd} Coplin jar (jar #2), with the present Washing Solution 2 at 42° C. for 5 minutes, into a 3^{rd} Coplin jar (jar #3) with the same solution at 42° C. for 5 minutes, to a 4^{th} Coplin jar (jar #4) with the Washing Solution 3 at room temperature for 5 minutes, to a 5^{th} Coplin jar (jar #5) with the Washing Solution 3 at room temperature for 5 minutes, to a 6^{th} Coplin jar (jar #6) with the diluted the Washing Solution 3 at room temperature for 2 minutes. The slide is finally pin-dried at 2000 rpm at 25° C. for 1 minute. The slide was stored in the dark before imaging.

(6) Data Acquisition and Analysis

For imaging the microarrays are imaged using a GenePix 4000A Array Scanner at Cy5 and C3 channel using two different sets of PMTs. Low PMT setting, where the brightest spot was close to saturation (65000 RFU) for each channel (Cy3 and Cy5). High PMT setting, where the top ˜5% of spots were saturated (>65000 RFU) for each color. All images are analyzed using the GenePix Pro 3.0 analysis software (Axon Instruments, Inc., Foster City, Calif.).

3. Assay Results and Array Performance

(1) High Sensitivity of Gene Expression Profiling

The sensitivity of assay is examined by hybridizing different amounts RNA to the human 2k cancer arrays. Varying amounts (0.5-5 μg) of total RNA from breast cancer cells MCF-7 are labeled with Cy3 (untreated cells) and Cy5 (the cancer cells treated with vitamin D3 for 6 hours). In FIG. 2, fluorescent image A represents the microarray after treatment with ˜5.0 μg RNA from an untreated MCF breast cancer cells, following a self-self hybridization. Fluorescent image B presents another similar microarray after treatment with ˜5.0 μg RNA from vitamin D-treated MCF breast cancer cells in Cy5-channel relative to RNA from untreated cells in Cy3-channel. The difference in expression profiles shown in the images A and B, are presented in graphs C and D, respectively. The results confirm that treatment of the cancer cells with vitamin D lead to the up-regulation of vitamin D-24 hydroxylase gene, while on the control slide after self-self hybridization from samples without treatment, no marker genes detected. Furthermore, hybridization results also show that the gene expression profile remained quite consistent with RNA from 0.5 μg to 5 μg under improved assay conditions. The up-regulation of vitamin D24 hydroxylase by vitamin D3 is observed repeatedly even with 0.5 μg of total RNA (data not shown).

(2) High-Sensitivity of Gene-Copy Detection

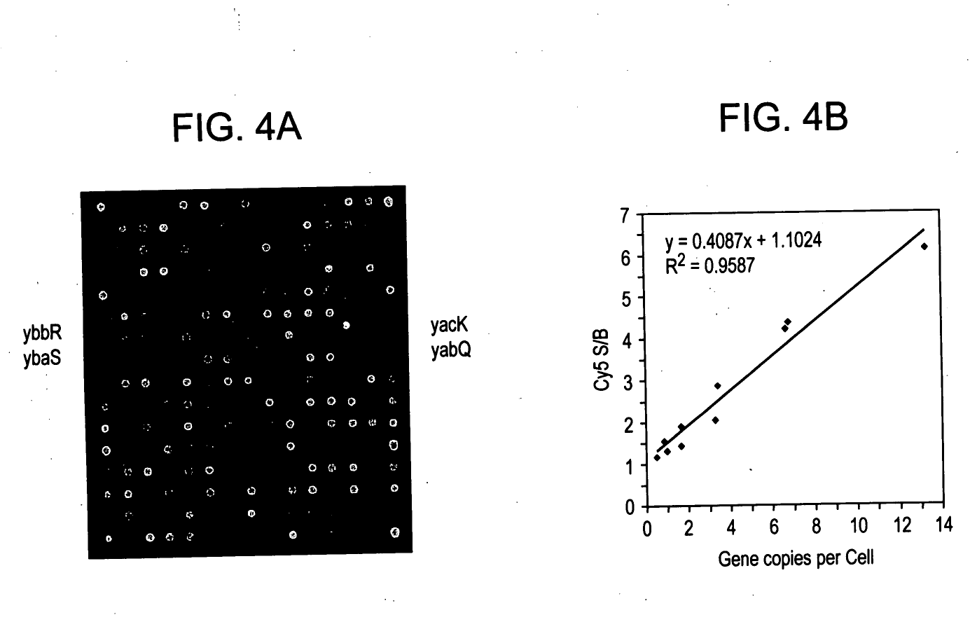

The detection limit of gene copy number in a sample is a common industrial standard to evaluate the sensitivity of the assays. Bacterial gene spiking experiment is used to serve this purpose. In the Corning human 10k array, a number of bacterial genes are also included in the same array to facilitate the assessment of microarray performance. Using pre-labeled serial dilution of bacterial target sequences, spiked into complex hybridization of labeled human RNA, the low limit of the gene copy number can be examined. FIG. 4 shows the results using the bacterial gene spiking experiment on a human 10K array. Different amounts (1 μg, 0.5 μg, 0.25μg, 0.125 μg and 0.075 μg) of in vitro transcripts of bacteria genes (yabQ, yacK, ybaS, and ybbR) labeled with Cy5 dye are spiked into a background of Cy3-labeled human brain and Cy5-labeled human testis cDNA generated from 4-5 μg of total RNA. A specified amount of labeled cDNA is added for hybridization; typically added based on the size of the glass coverslip used for hybridization. The amount of labeled cDNA corresponds to a pmol value as calculated from optical density measures of the labeled cDNA. (See, FIGS. 14A-F for the calculations and procedures.) For example 36-50 pmol of labeled cDNA is used for hybridization when using a 24×60 mm glass coverslip. Uniform addition of labeled cDNA per hybridization has a great influence on reproducibility, consistency, low coefficients of variation, no non-specific hybridization, and background control. The quality and consistency of the labeled cDNA material added for hybridization should be tightly controlled. Fluorescent image A presents one subgrid of the 10K array after hybridization. The graph B presents a plot of Cy5 signal-to background ratio versus gene copy number per cell. Results indicate that the sensitivity of the assays performed using the present reagent kits is better than one copy in 0.5×10⁶ cells, which is about 5-10 folder better than leading competitive kits.

(3) Assay Reproducibility

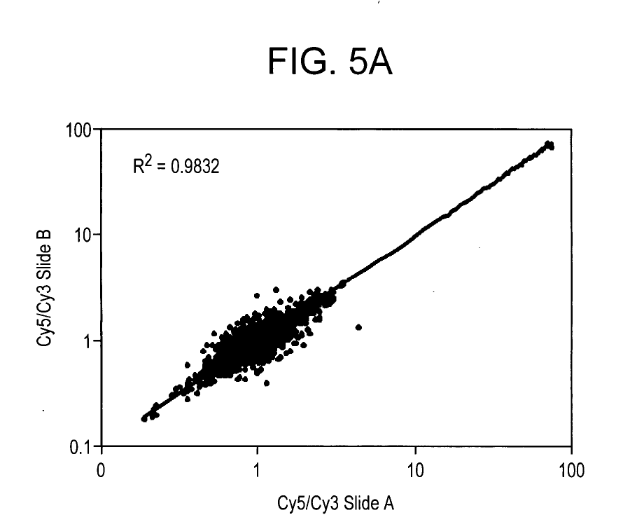

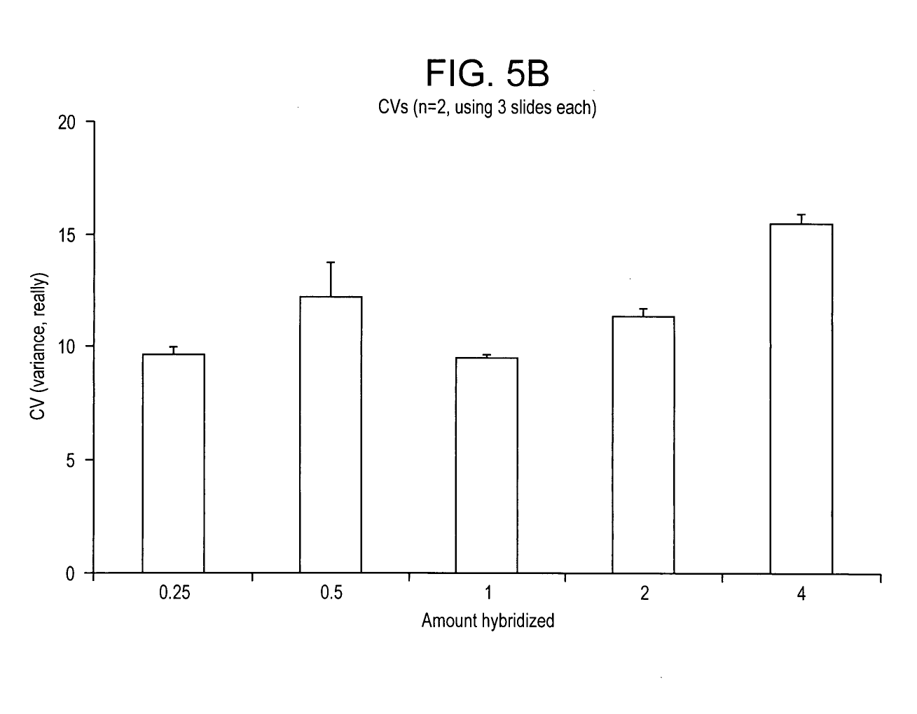

FIG. 5 is a demonstration of high reproducibility of a gene expression profile, according to the present invention, using a human 2K cancer array. The graph shows the ratio of Cy5/Cy3 for RNA from D3-treated MCF cells between two slides, each having duplicate subarrays. The median variance of the ratio is about 5-6% between the slides or between subarrays on the same slide. This low median variance in the assay results suggests the high reproducibility of the assays using the reagent system of the present invention.

The present invention has been described in general and in detail by way of examples. Persons skilled in the art understand that the invention is not limited necessarily to the specific embodiments disclosed. Modifications and variations may be made without departing from the scope of the invention as defined by the following claims or their equivalents, including equivalent components presently known, or to be developed, which may be used within the scope of the present invention. Hence, unless changes otherwise depart from the scope of the invention, the changes should be construed as being included herein.

1. A reagent system for gene analysis or expression assays using a nucleic acid microarray, the system comprising:
a) a probe spotting solution containing about 30-96% vol. of an aqueous medium comprising dimethylsulfoxide (DMSO), ethylene glycol (EG), or a combination thereof, a buffer with a pH value of about 3.5-9.5, water, and nucleic acid;
b) a probe-labeling buffer composition containing random oligonucleotide hexamers, optionally with oligonucleotide dT primers, in a RNAse and DNAse free-aqueous medium;
c) a pre-hybridization solution containing a blocker reagent to reduce non-specific binding of targets to surface or probes;
d) a hybridization solution comprising about: 0.1-5% of a water soluble protein, 20-70% vol. formamide, optionally with either 0.05-1.5% of a surfactant or less than about 10% dextran sulfate, or both; and
e) optionally a background-reducing solution containing about 0.01-1% wt. of a borohydride salt.

2. The reagent system according to claim 1, further includes a wash reagent A, comprising a buffered solution with a pH of 7.0±0.15, or a wash reagent B, comprising a buffered solution containing a surfactant with a pH of 5.5±0.15.

3. The reagent system according to claim 1, wherein said system is an assembly of at least one reagent solution, containing at least one component, wherein constituent components are said solution are premixed and stored in a single container.

4. A reagent system for gene expression assays using probe nucleic acid microarrays, the system comprising:
a) a printing kit, comprising a nucleic acid spotting solution; and
b) a hybridization kit, comprising: a target-labeling solution, a nucleic acid pre-hybridization solution, a nucleic acid hybridization solution, and optionally a background-reducing solution, wherein constituent components of said printing and hybridization kits are stable and retain functional performance, when stored together at a temperature between about −20° C. to about 60° C.

5. The reagent system according to claim 4, wherein said constituent components of said printing and hybridization kits are stable and retain functional performance when stored at a temperature between about 10° C. to about 50° C.

6. The reagent system according to claim 4, wherein components of said reagent system are stable and retain functional performance when stored at a temperature between about 15° C. to about 45° C.

7. The reagent system according to claim 4, wherein constituent components of said pre-hybridization solution are each premixed and stored in a single container.

8. The reagent system according to claim 4, wherein said system further comprises a number of planar substrates having an amine-reactive surface.

9. The reagent system according to claim 8, wherein said planar substrates having an γ-aminopropylsilane-coated surface.

10. The reagent system according to claim 8, wherein said planar substrates are flat glass slides of a non-sodium borosilicate composition.

11. The reagent system according to claim 4, wherein said nucleic acid spotting solution comprises about 30% to about 95% volume dimethylsulfoxide (DMSO) or ethylene glycol in a pH buffer solution, when said probe nucleic acid in said microarrays is either cDNA or oligonucleotide.

12. The reagent system according to claim 10, wherein said pH buffer solution has a pH of ˜4-10, when prepared with either acetate, citrate, citrate-phosphate, maleate, or succinate.

13. The reagent system according to claim 4, wherein said spotting solution comprises about 30% to about 95% volume ethylene glycol, optionally with 10-50% formamide, in a pH buffer solution, when said probe nucleic acid on said microarrays is cDNA.

14. The reagent system according to claim 4, wherein said spotting solution comprises about 1% to about 55% by volume of ethylene glycol or formamide either individually, together in combination, or with DMSO in a pH buffer solution.

15. The reagent system according to claim 4, wherein said target-labeling solution comprises a random selection of oligonucleotides, either hexamers (6mers) or 9mers when labeling mRNA.

16. The reagent system according to claim 4, wherein said target-labeling solution comprises a random selection of oligonucleotides, either hexamers (6mers) or 9mers, together with an oligonucleotide dT primer, in RNAse- or DNAse-less aqueous solution, when labeling total RNA.

17. The reagent system according to claim 4, wherein said background reducing solution contains BH4^{- and} 1×SSC.

18. The reagent system according to claim 4, wherein said pre-hybridization solution comprises about: 20-70% vol. formamide, 0.1-5% wt. aqueous-soluble protein in buffer solution of 2×SSC (about 300 mM sodium chloride, 30 mM sodium citrate) at pH of about 6-9, when the said solution is applied to a cDNA microarray

19. The reagent system according to claim 4, wherein said pre-hybridization solution comprises about: 40-60% formamide, 0.2-1.7% aqueous-soluble protein in buffer solution of 2×SSC at pH of about 7-8, when said solution is applied to an oligonucleotide microarray.

20. The reagent system according to claim 18 or 19, wherein said aqueous-soluble protein is a low-fluorescence bovine serum albumin.

21. The reagent system according to claim 4, wherein said pre-hybridization solution comprises about: 0.1-5% wt. aqueous-soluble protein, 0.05-5% vol. of a surfactant in buffer solution of 2×SSC at pH of about 6-9, when said solution is applied to either a cDNA or oligonucleotide microarray.

22. The reagent system according to claim 22, wherein said pre-hybridization solution comprises about: 0.2-1.7% aqueous soluble protein, 0.1-1.5% vol. sodium lauryl sulfate in buffer solution of 2×SSC at pH of about 7-8.

23. The reagent system according to claim 4, wherein said hybridization solution comprises about: 20-70% vol. formamide, 0.05-1.5% of a surfactant, 0.1-5% wt. aqueous-soluble protein, 1-10% dextran sulfate, 0.01-0.5 mg/ml poly-A, 0.1-50 μg/ml Cot-1 DNA, in buffer solution of 0.5-7×SSC at pH of about 6-9.

24. The reagent system according to claim 22, wherein said hybridization solution composition includes about: 40-60% formamide, 0.05-0.5% sodium lauryl sulfate, 0.1-1.5% wt. aqueous soluble protein, 2-7% dextran sulfate, 0.05-0.25 mg/ml poly-A, 1-12 μg/ml Cot-1 DNA, in buffer solution of 0.5-2×SSC at pH of about 6.5-7.5.

25. The reagent system according to claim 4, further comprising a wash reagent A that contains about 20×SSC (3M sodium chloride, 0.3M sodium citrate-2H₂O), at a pH of about 7.0.

26. The reagent system according to claim 4, further comprising a wash reagent B that contains about 10% surfactant in aqueous solution, at a pH of 5.5±0.15.

27. A method for performing a biological array on a nucleic acid microarray, the method comprises:
a) providing a reagent system comprising:
i) a probe spotting solution containing about 30-96% vol. of an aqueous medium comprising dimethylsulfoxide (DMSO), ethylene glycol (EG), or a combination thereof, a buffer with a pH value of about 3.5-9.5, water, and nucleic acid;
ii) a probe-labeling buffer composition containing random oligonucleotide hexamers, optionally with oligonucleotide dT primers, in a RNAse and DNAse free-aqueous medium;
iii) a pre-hybridization solution containing a blocker reagent to reduce non-specific binding of targets to surface or probes;
iv) a hybridization solution comprising about: 0.1-5% of a water soluble protein, 20-70% vol. formamide, optionally with either 0.05-1.5% of a surfactant or less than about 10% dextran sulfate, or both; and
v) optionally a background-reducing solution containing about 0.01-1% wt. of a borohydride salt;
b) reformulating nucleic acid sequences with said probe spotting solution to a final concentration;
c) preparing dye-labeled target sequences with said target-labeling solution in combination with reverse transcription system reagents;
d) treating a probe-bearing substrate with said pre-hybridization solution;
e) optionally treating said probe-bearing substrate with said background-reducing solution;
f) applying quantified target sequences to said probe-bearing substrate, and allow said target sequences to hybridize with probe sequences.

28. The method according to claim 27, wherein said probe sequences are at specific pmol concentrations and temperatures in a specified volume based on the size of a coverglass used for hybridization.

28. The method according to claim 27, wherein said method further includes following target-labeling purification protocols for determining amounts of primers and nucleotides for labeling; and quantifying and characterizing labeled cDNA.

29. The method according to claim 28, wherein said target-labeling purification protocols incorporates the use of RNAse A and H, followed by ethanol purification and column purification.

30. The method according to claim 27, wherein said reagent system further comprises a wash reagent A, comprising a buffered solution with a pH of 7.0±0.15, or a wash reagent B, comprising a buffered solution containing a surfactant with a pH of 5.5±0.15.

31. The method according to claim 27, further comprising preparing three wash solutions 1, 2, and 3, from wash reagents A and B, respectively.

32. The method according to claim 31, wherein said 1) wash solution 1 includes: deionized water, wash reagent A and wash reagent B; 2) wash solution 2 includes: deionized water and wash reagent A; 3) wash solution 3 includes: wash solution 2 and deionized water.

33. The method according to claim 31, further comprising washing said microarray with said washing solutions 1, 2 and 3, either in a sequential manner or combined together.

34. The method according to claim 27, wherein said final concentration of said probe spotting solution is about 0.05-1 mg/ml.
